(19) United States
(12) Patent Application Publication
(10) Pub. No.: US 2004/0142343 A1
(43) Pub. Date: Jul. 22, 2004

(54) Helicobacter bizzozeronii urease genes and their uses in diagnostic and treatment methods

(76) Inventors: Yung-Fu Chang, Ithaca, NY (US); Kenneth W. Simpson, Ithaca, NY (US); Jiaqian Zhu, Ithaca, NY (US)

Correspondence Address: Michael L. Goldman, Esq., NIXON PEABODY LLP, Clinton Square, P.O. Box 31051, Rochester, NY, 14603-1051, US

(21) Appl. No.: 10/639,273

(22) Filed: Aug. 12, 2003

Related U.S. Application Data
(60) Provisional application No. 60/404,337, filed on Aug. 16, 2002.

(51) Int. Cl. C12Q 1/68; C07H 21/04; C12N 9/78; C12N 1/21

(52) U.S. Cl. 435/6; 435/69.1; 435/227; 435/252.3; 435/320.1; 536/23.2

(57) ABSTRACT

The present invention relates to an isolated nucleic acid molecule conferring on Helicobacter bizzozeronii an ability to produce urease. The present invention also relates to the isolation of two urease structural genes, ureA and ureB, and five urease accessory genes, ureE, ureF, ureG, ureH, and ureI, of H. bizzozeronii. Also disclosed are deduced protein and polypeptide sequences of the nucleic acid molecules and genes of the present invention. Detection and treatment methods relating to H. bizzozeronii are also disclosed.

This application claims the benefit of U.S. Provisional Patent Application Serial No. 60/404,337, filed Aug. 16, 2002.

### FIELD OF THE INVENTION

The present invention relates to an isolated nucleic acid molecule corresponding to a urease gene cluster of Helicobacter bizzozeronii and to the urease genes located within the urease gene cluster, as well as to the use of the nucleic acid molecules and their corresponding proteins or polypeptides in drugs, vaccines, and diagnostic tests.

### BACKGROUND OF THE INVENTION

Helicobacter bizzozeronii is a spiral, Gram-negative microaerophilic bacterium that was first cultured and characterized from gastric biopsies of canines (Hanninen et al., “Culture and Characteristics of Helicobacter bizzozeronii, a New Canine Gastric Helicobacter sp.,” Int. J. Syst. Bacteriol. 46:160-6 (1996)). H. bizzozeronii is 5 to 10 μm long and 0.3 μm wide with bipolar sheathed flagella and is indistinguishable morphologically from the Lockard type 3 bacterium and from Helicobacter heilmanii (Solnick et al., “Emergence of Diverse Helicobacter Species in the Pathogenesis of Gastric and Enterohepatic Diseases,” Clin. Microbiol. Rev. 14(1):59-97 (2001)). H. bizzozeronii is a slow growing organism whose colonies can be seen clearly on blood agar after about 8 to 10 days of incubation.

Although there are differences between H. bizzozeronii and other Helicobacter species, the phenotypic characteristics of the only human H. heilmanii isolate (R-53) to date are consistent with those of H. bizzozeronii, H. felis, and H. salomonis (Jalava et al., “A Cultured Strain of “Helicobacter heilmannii,” a Human Gastric Pathogen, Identified as H. bizzozeronii: Evidence for Zoonotic Potential of Helicobacter,” Emerg. Infect. Dis. 7:1036-1038 (2001)). H. bizzozeronii and other Helicobacter spp. are potential zoonotic agents (Jalava et al., “A Cultured Strain of “Helicobacter heilmannii,” a Human Gastric Pathogen, Identified as H. bizzozeronii: Evidence for Zoonotic Potential of Helicobacter,” Emerg. Infect. Dis. 7:1036-1038 (2001); Simpson et al., “The Relationship of Helicobacter spp. Infection to Gastric Disease in Dogs and Cats,” J. Vet. Intern. Med. 14:223-227 (2000); Simpson et al., “Helicobacter felis Infection in Dogs: Effect on Gastric Structure and Function,” Vet. Pathol. 36:237-248 (1999); Simpson et al., “Gastric Function in Dogs with Naturally Acquired Gastric Helicobacter spp. Infection,” J. Vet. Intern. Med. 13:507-515 (1999); Simpson et al., “Helicobacter felis Infection is Associated with Lymphoid Follicular Hyperplasia and Mild Gastritis but Normal Gastric Secretory Function in Cats,” Infect. Immun. 68:779-790 (2000); Simpson et al., “Helicobacter pylori Infection in the Cat: Evaluation of Gastric Colonization, Inflammation and Function,” Helicobacter 6:1-14 (2001); and Strauss-Ayali et al., “Gastric Helicobacter Infection in Dogs,” Vet. Clin. North. Am. Small. Anim. Pract. 29:397-414, vi (1999)). Furthermore, infection with H. heilmanii may be more frequently mucosa associated lymphoid tissue (MALT) lymphoma (Anderson et al., “Characterization of a Culturable “Gastrospirillum hominis” (Helicobacter heilmannii) Strain Isolated from Human Gastric Mucosa,” J. Clin. Microbiol. 37:1069-1076 (1999).

Helicobacter species are known to play a role in gastric diseases such as ulcers and antral gastritis in both humans and animals. Due in part to the lack of definitive detection and diagnostic tools, H. bizzozeronii has not been studied as much as other Helicobacter species such as H. pylori, which is responsible for chronic active gastritis in people and animals (Solnick et al., “Emergence of Diverse Helicobacter Species in the Pathogenesis of Gastric and Enterohepatic Diseases,” Clin. Microbiol. Rev. 14(1):59-97 (2001)). Thus, knowledge of other Helicobacter species is important in understanding the role that H. bizzozeronii may play in various gastric conditions of canines and other animals, as well as in the development of diagnostic and treatment tools for the bacterium.

In H. pylori, several virulence factors, including CagA, VacA, and urease have been identified (Figura et al., “Helicobacter pylori caga and vacA Types and Gastric Carcinoma,” Dig. Liver Dis. 32 Supp. 3:S182-3 (2000); and Labigne et al., “Shuttle Cloning and Nucleotide Sequences of Helicobacter pylori Genes Responsible for Urease Activity,” J. Bacteriol. 173(6):1920-31 (1991)).

Urease is a heteromultimer nickel-containing metalloenzyme (Mobley et al., “Molecular Biology of Microbial Ureases,” Microbiol. Rev. 59:451-480 (1995)). Urease activity is very important in gastric colonization because it breaks down ureA to generate NH₃ which neutralizes gastric acid and protects the bacterium until it enters the protective barrier of the gastric mucus. Thus, it facilitates bacterial survival in an acidic environment.

Two of the urease structural subunit genes, ureA and ureB, and five accessory genes were identified in H. pylori and H. hepaticus (Beckwith et al., “Cloning, Expression, and Catalytic Activity of H. hepaticus Urease,” Infect. Immun. 69:5914-5920 (2001); Mobley et al., “Molecular Biology of Microbial Ureases,” Microbiol. Rev. 59:451-480 (1995); Olson et al., “Requirement of Nickel Metabolism Proteins HypA and HypB for Full Activity of Both Hydrogenase and Urease in Helicobacter pylori,” Mol. Microbiol. 39:176-182 (2001); and van Vliet et al., “Nickel-Responsive Induction of Urease Expression in Helicobacter pylori is Mediated at the Transcriptional Level,” Infect. Immun. 69:4891-4897 (2001)). The urease structural subunit genes, urea and ureB, of H. felis, H. heilmanii, H. mustelae, and H. hepaticus have been cloned and sequenced (Beckwith et al., “Cloning, Expression, and Catalytic Activity of H. hepaticus Urease,” Infect. Immun. 69:5914-5920 (2001); Ferrero et al., “Cloning, Expression and Sequencing of Helicobacter felis Urease Genes,” Mol. Microbiol. 9:323-333 (1993); Labigne et al., “Shuttle Cloning and Nucleotide Sequences of Helicobacter pylori Genes Responsible for Urease Activity,” J. Bacteriol. 173:1920-1931 (1991); Solnick et al., “Construction and Characterization of an Isogenic Urease-Negative Mutant of Helicobacter mustelae,” Infect. Immun. 63:3718-3721 (1995)). The accessory genes are responsible for incorporation of nickel ions into the UreB protein and activation of the enzyme (Mobley et al., “Molecular Biology of Microbial Ureases,” Microbiol. Rev. 59:451-480 (1995); and Olson et al., “Requirement of Nickel Metabolism Proteins HypA and HypB for Full Activity of Both Hydrogenase and Urease in Helicobacter pylori,” Mol. Microbiol. 39:176-182 (2001)). UreI is essential for bacterial survival in a low pH environment (Rektorschek et al., “Acid Resistance of Helicobacter pylori Depends on the UreI Membrane Protein and an Inner Membrane Proton Barrier,” Mol. Microbiol. 36:141-152 (2000); and Skouloubris et al., “The Helicobacter pylori UreI Protein is Not Involved in Urease Activity but is Essential for Bacterial Survival In Vivo,” Infect. Immun. 66:4517-4521 (1998)).

Ammonia generated from ureA by urease accelerates an apoptosis induced by tumor necrosis factor (TNF-α) in gastric and duodenal epithelial cells. Therefore, urease is suggested to play a crucial role in the development of ulcers in the stomach and the duodenum (Andrutis et al., “Inability of An Isogenic Urease-Negative Mutant Strain of Helicobacter mustelae to Colonize the Ferret Stomach,” Infect. Immun. 63:3722-3725 (1995); Eaton et al., “In Vivo Complementation of ureB Restores the Ability of Helicobacter pylori to Colonize,” Infect. Immun. 70:771-778 (2002); Fan et al., “Helicobacter pylori Urease Binds to Class II MHC on Gastric Epithelial Cells and Induces their Apoptosis,” J. Immunol. 165:1918-1924 (2000); Igarashi et al., “Ammonia as an Accelerator of Tumor Necrosis Factor Alpha-Induced Apoptosis of Gastric Epithelial Cells in Helicobacter pylori Infection,” Infect. Immun. 69:816-821 (2001); Kohda et al., “Role of Apoptosis Induced by Helicobacter pylori Infection in the Development of Duodenal Ulcer,” Gut. 44:456-462 (1999); and Smoot et al., “Helicobacter pylori Urease Activity is Toxic to Human Gastric Epithelial Cells,” Infect. Immun. 58:1992-1994 (1990)).

Urease has also been reported to be a virulence factor of Helicobacter spp., and is essential for colonization of the stomach (Mobley et al., “Helicobacter pylori Urease: Properties and Role in Pathogenesis. Scand. J. Gastroenterol. Suppl. 187:39-46 (1991); Mobley et al., “Molecular Biology of Microbial Ureases,” Microbiol. Rev. 59:451-480 (1995); Tsuda et al., “Essential Role of Helicobacter pylori Urease in Gastric Colonization: Definite Proof Using a Urease-Negative Mutant Constructed by Gene Replacement,” Eur. J. Gastroenterol. Hepatol. 6(Suppl 1):S49-52 (1994); and Tsuda et al., “A Urease-Negative Mutant of Helicobacter pylori Constructed by Allelic Exchange Mutagenesis Lacks the Ability to Colonize the Nude Mouse Stomach,” Infect. Immun. 62:3586-3589 (1994)). As with H. pylori and other gastric Helicobacter species, H. bizzozeronii has been shown to express a high level of urease.

The importance of urease as a virulence factor has been demonstrated in several Helicobacter species, which can be illustrated using H. pylori. Like most bacteria, H. pylori is sensitive to a medium of acidic pH, although it can tolerate acidity in the presence of physiological levels of urea (Marshall et al., “Urea Protects Helicobacter (Campylobacter) pylori from the Bactericidal Effect of Acid,” Gastroenterology 99(3):697-702 (1990)). By hydrolysing ureA to carbon dioxide and ammonia, which are released into the microenvironment of the bacterium, the urease of H. pylori is assumed to permit the survival of the bacterium in the acidic environment of the stomach. Thus, urease activity is very important in gastric colonization of bacteria and facilitation of bacteria survival in an acidic environment. Urease also plays an important role in causation of epithelium cell apoptosis, because it breaks down the ureA to generate NH₃, which could immediately neutralize the gastric acid surrounding the bacterium until it enters the protective barrier of the gastric mucus (Andrutis et al., “Inability of An Isogenic Urease-Negative Mutant Stain of Helicobacter mustelae to Colonize the Ferret Stomach,” Infect. Immun. 63(9):3722-5 (1995); Tsuda et al., “Essential Role of Helicobacter pylori Urease in Gastric Colonization: Definite Proof Using a Urease-Negative Mutant Constructed By Gene Replacement,” Eur. J. Gastroenterol. Hepatol. 6 Supp. 1:S49-52 (1994)). Studies conducted on animal models have provided elements suggesting that urease is an important factor in the colonization of the gastric mucosa (Eaton et al., “Essential Role of Urease in Pathogenesis of Gastritis Induced By Helicobacter pylori in Gnotobiotic Piglets,” Infect. Immun. 59(7):2470-5 (1991)).

Urease is also suspected of causing injury either directly or indirectly to the gastric mucosa. H. pylori is presently recognized as the etiological agent of antral gastritis, and appears to be one of the cofactors required for the development of ulcers. Furthermore, it seems that the development of gastric carcinomas may be linked to the presence of H. pylori.

In addition to this role in the colonization of the stomach, urease, along with the ammonia released, might have a direct cytotoxic effect on epithelial cells and an indirect effect by inducing an inflammatory response which might be responsible for the gastric lesions. Thus, urease is one of the most important determinants of pathogenicity of a particular strain of Helicobacter. Further, the construction of isogenic strains of Helicobacter known to cause gastric conditions in animals and humans may be used to develop ways of inactivating the genes responsible for the expression of urease.

Therefore, identifying the genes responsible for the production and activation of urease in H. bizzozeronii is an important step in developing detection assays, diagnostic procedures, and treatment protocols for this bacterium in animals such as canines and felines.

The present invention is directed to overcoming the deficiencies in the prior art.

### SUMMARY OF THE INVENTION

The present invention relates to an isolated nucleic acid molecule conferring on Helicobacter bizzozeronii an ability to produce urease. The nucleic acid molecule is a urease gene cluster having at least one urease structural gene and at least one urease accessory gene.

The present invention also relates to an isolated nucleic acid molecule from a Helicobacter bizzozeronii urease gene cluster, where the nucleic acid molecule is one of the following genes located in SEQ ID NO: 1: ureA; ureB; ureE; ureF; ureG; ureH; and ureI.

In addition to the various isolated nucleic acid molecules described above, the present invention relates to isolated proteins or polypeptides encoded by those isolated nucleic acid molecules. The isolated nucleic acid molecules can be inserted as heterologous DNA in an expression vector forming a recombinant DNA expression system for producing the proteins or polypeptides. Likewise, the heterologous DNA, usually inserted in an expression vector to form a recombinant DNA expression system, can be incorporated in a cell to achieve this objective.

The isolated proteins or polypeptides of the present invention can be combined with a pharmaceutically-acceptable carrier to form a vaccine or used alone for administration to mammals, for preventing onset of disease resulting from infection by Helicobacter bizzozeronii. Alternatively, each of the proteins or polypeptides of the present invention can be used to raise an antibody or a binding portion thereof. The antibody or binding portion thereof may be used alone or combined with a pharmaceutically-acceptable carrier to treat mammals already exposed to Helicobacter bizzozeronii to induce a passive immunity to prevent disease occurrence.

The proteins or polypeptides of the present invention or the antibodies or binding portions thereof raised against them can also be utilized in a method for detection of Helicobacter bizzozeronii in a sample of tissue or body fluids. When the proteins or polypeptides are utilized, they are provided as an antigen. Any reaction with the antigen or the antibody is detected using an assay system which indicates the presence of Helicobacter bizzozeronii in the sample. Alternatively, Helicobacter bizzozeronii can be detected in such a sample by providing a nucleotide sequence of the isolated nucleic acid molecules of the present invention as a probe in a nucleic acid hybridization assay or a gene amplification detection procedure (e.g., using a polymerase chain reaction procedure). Any reaction with the probe is detected so that the presence of Helicobacter bizzozeronii in the sample is indicated.

Isolation of the nucleic acid molecules of the present invention constitutes a significant advance in the treatment and detection of such bacteria. It also provides the basis for a vaccine to prevent infection by Helicobacter bizzozeronii and a pharmaceutical agent for passive immunization for those mammals exposed to Helicobacter bizzozeronii. The proteins utilized in the vaccine, or used to produce the pharmaceutical agent, can be produced at high levels using recombinant DNA technology.

In diagnostic applications, the proteins or polypeptides of the present invention, as well as antibodies and binding portions thereof against them, permit rapid determination of whether a particular individual mammal is infected with Helicobacter bizzozeronii. Moreover, such detection can be carried out without requiring an examination of the individual mammal being tested for an antibody response.

### BRIEF DESCRIPTION OF THE DRAWINGS

FIG. 1 shows the restriction enzyme maps of the Helicobacter bizzozeronii urease clones. The urease structural genes ureA and ureB are identified as A and B, respectively. The urease accessory genes ureI, ureE, ureF, ureG, and ureH are identified as I, E, F, G, and H, respectively. Within the restriction maps, the restriction sites are identified as follows: B: BamHI; Ha: HaeI; H: HindIII; N: NdeI; P: PstI; and X: XbaI.

FIGS. 2A-2C show the nucleotide sequence (SEQ ID NO: 1) of the ureABIEFGH gene cluster and its upstream region and the predicted amino acid sequences of the putative S-Adenosylmethionine: tRNA Ribosyltransferase (SEQ ID NO:31); the Glucose Inhibited Division Protein B (SEQ ID NO:32); the hypothetical protein (SEQ ID NO:33); and the UreA (SEQ ID NO:3), UreB (SEQ ID NO:5), UreI (SEQ ID NO:15), UreE (SEQ ID NO:7), UreF (SEQ ID NO:9), UreG (SEQ ID NO:11), and UreH (SEQ ID NO:13) proteins. Promoter-like regions proximal to ureA and ureI genes are indicated by bold and italics. Potential ribosome binding sequences preceeding ureA and ureI are underlined, and the transcription start site of ureA and ureI are bolded. A potential transcription terminator is indicated by ------> <-----.

FIGS. 3A-3C show the alignment of the predicted amino acid sequences of UreA (FIG. 3A) and UreB (FIGS. 3B-3C). Amino acid sequences were predicted from the known nucleotide sequences of UreAB (H. bizzozeronii, H. b: AB; H. heilmannii, H. he: AB (Genbank number, AF329400); H. felis, H. f: AB (Genbank number, X69080); H. pylori, H. p: AB (Genbank number, X57132); H. hepaticus, H. hep: AB (Genbank number, AF332654); and H. mustelae, H. m.: AB (Genbank number, L33462). FIG. 3A: The predicted amino acid sequences for the UreA proteins are denoted as follows (source organisms are identified in parentheses): H. b: A (H. bizzozeronii) (SEQ ID NO:3); H. he: A (H. heilmannii) (SEQ ID NO:34); H. f: A (H. felis) (SEQ ID NO:35); H. p: A (H. pylori) (SEQ ID NO: 36); and H. hep: A (H. hepaticus) (SEQ ID NO: 37). FIGS. 3B-3C: The predicted amino acid sequences for the UreB proteins are denoted as follows (source organisms are identified in parentheses): H. b: B (H. bizzozeronii) (SEQ ID NO:5); H. he: B (H. heilmannii) (SEQ ID NO: 38); H. f: B (H. felis) (SEQ ID NO: 39); H. p: B (H. pylori) (SEQ ID NO:40); and H. hep: B (H. hepaticus) (SEQ ID NO: 41).

FIG. 4 shows the Western blot analysis of whole-cell extracts of H. bizzozeronii, H. felis, and E. coli Topo10 cells harboring the recombinant plasmids using rabbit antiserum raised against H. bizzozeronii UreB. Each lane was loaded with 8 μL of sample. The sample was prepared from the pellet of 1 mL of culture that was induced with 1 mM IPTG at OD₆₀₀ nm=0.5 and incubated until OD₆₀₀ nm=2.5. The pellet was suspended in 200 μL sample buffer, boiled for 5 minutes, and centrifuged for 2 minutes before loading. Lane 1: Vector control (pBluescript). Lane 2: Clone (pHB1). Lane 3: Clone 2 (pHB2). Lane 4: Clone 3 (pHB3). Lane 5: Clone 4 (pHB4). Lane 6: Clone 5 (pHB5). Lane 7: H. bizzozeronii. Lane 8: Clone (pKK223-3-UreB). Lane 9: H. felis.

FIG. 5 shows the Western blot analysis of H. bizzozeronii rUreB against sera from cats infected with Helicobacter-like strains. Lanes 1, 2, and 3: Serum from uninfected cat. Lanes 4, 5, 6, 7 and 8: Sera from Helicobacter-like infected cats.

FIG. 6 shows the Western blot analysis of H. bizzozeronii UreB against sera from dogs infected with Helicobacter-like strains. Lanes 1, 2, and 3: Sera from uninfected dogs. Lanes 4, 5, 6, 7, and 8: Sera from Helicobacter-like infected dogs.

FIG. 7 shows the urease activity in H. bizzozeronii and recombinant plasmids pHB1, pHB2, pHB3, pHB4, and pHB5, and a vector control in E. coli (pbs). The background activity of the vector control was subtracted from the activity of each recombinant plasmid.

### DETAILED DESCRIPTION OF THE INVENTION

One aspect of the present invention relates to an isolated nucleic acid molecule conferring on Helicobacter bizzozeronii an ability to produce urease. This nucleic acid molecule is a urease gene cluster having at least one urease structural gene and at least one urease accessory gene.

In one embodiment, the urease gene cluster comprises the nucleotide sequence corresponding to SEQ ID NO: 1 as follows:

| | |
|---|---|
| aattaatggt ctatgagcgt gccagcaaca aactcaccca cagcactttt aaagaaatct | 60 |
| ttgatttttt tcccccccat gcccttgtgg ttctcaatga taccaaagtg atcaaagcac | 120 |
| ggctctttgc tcacaccgcc caaaaacgcc cagtagagat tttattacac cacgcgacta | 180 |
| gccccacagc gtgtttagcc caaatgcgcg ggaaagtgcg ccccggactc gtgctggggt | 240 |
| tagagggggg ttattcttgt gaggtcttag aagtgctaga taatggtttg agggtgttaa | 300 |
| gtttcaaaca tgggggagag cttttagagt gggcgggggt tttagagatg ttggaactct | 360 |
| tagggcatgt gcccctccca ccttatttaa aacgccccga tgaggcgcaa gatttgcaag | 420 |
| attaccaaag cgtctttgcc aaataccccg gagccatcgc tgcgcccacc gcatctttac | 480 |
| atttctcaca gagcgcaaag gaccacctat taaagaattt taagcatgtt tttatcactt | 540 |
| tgcatgtggg ggcggggaca tttttaagcg tgcagagtga ggacattaga gaacacccca | 600 |
| tgcatgctga attttgtaga agtctcccac aagccgggct taccctagat gaagcccctt | 660 |
| atattttatg tgtgggcacc accgctttaa gagccacaga gcattacaaa aggggttttg | 720 |
| ggcttaaacc ttgcgaactc tttttgcacc taggcaaccc ggtcaagcac gcccaagccc | 780 |
| tgctgacaaa tttccacctg cccgaatcga ccttgatcat gctcgtagcc tctatggtgg | 840 |
| gcttggataa atgcctagaa ctctataaaa ttgccataga acaccactac cgcttttatt | 900 |
| cctatggcga tgggatgttg atcttatgag ggctaaatta gacctattta cccatttgct | 960 |
| tttagagtgg gggagcgtgc ataatttgag cggggcaaaa aaccaccaag atatagagca | 1020 |
| taatatccaa gatagcttgc aagtcttgga ctttatcgct ccctttaagt cttgcttgga | 1080 |
| tatagggagt ggggcgggtt ttccagccat ccccttaagc ctagcctgct ctaatgcccg | 1140 |
| ctttatctta ttagagccca atgctaaaaa agtggctttt ttgcaccatg tgaagcttgc | 1200 |
| cttgaacttg aataacctag aaatccaacg catgcggatc gaacatgtaa gcccccaaag | 1260 |
| tgtgttggtc gatctcatca cctctagagc cttgatgaac gcccaaaatt tgatcgcctt | 1320 |
| gagcgcgccc tttttaaggg agcaggggca ttttttattt tacaagggta gccatctgcg | 1380 |
| cactgagatc gcttgtgccg atcatgaatg ccatgtatat ggcaaacggg tttattttta | 1440 |
| ctcccaaagg agagattttg cttagattat taattccttt actcatcatt ggctggattt | 1500 |
| gctggctctt tttgcgcccc aaaaagagca aaccccgccc ttctaatcct tcccaaattg | 1560 |
| agagcctatt agagtgcgcg cattgccaaa cctatgtttc tagccaagag gcgtttttta | 1620 |
| gcaatgggcg tgcctattgt tgccaagcgt gcttgcaaaa aggggattca tgttagtttt | 1680 |
| tggacaccca caaatccctt gcatgccttt taaatgcgtg gctgatatac aggcgatcgc | 1740 |
| ccaagtgcct agccacacga tccccttttt ccaaacctgc cacacagacg cttttggact | 1800 |
| ttccaaacat tgtagcgatc aaggggtcaa atacgccgtg tgggtgcaaa accctttaga | 1860 |
| gtgcgcttta atggcaaatt ttaaaccgac atatatcttg gcggaaaacc aattagaggc | 1920 |
| atgccaaagc attgctaatg actacttgtt cgatgcgcgc attttaaagg tgattgagca | 1980 |
| cgcagagcaa ttagaagagg tgcttaaatt acgcctagat ggtgcagtgt tccgatcttt | 2040 |
| tttgtggctt tagagtgttt ttagcctcca tagattaaaa tagccctttt ggagagatgt | 2100 |
| ccgagtggtt gaaggagcac gcctggaacg cgtgtaaggt gcaagccttc gagggttcga | 2160 |
| atccctttct ctccgccatt ttcccctatt tttatgtttt aagccaataa gatcaactta | 2220 |
| gaataaataa tattatcttt gctaacgaaa atattaacaa aggttagcca aaacagacta | 2280 |
| gaatttgtcc cgttgatagc ctagctattc aataatacaa atttggtaga aggagtttag | 2340 |
| gatgaaatta acccctaaag agctggacaa gctcatgttg cattatgcgg gcgaattggc | 2400 |
| taaaaaacgc aaagcaaatg gcgttaagct aaattatact gaggcagtag ccctcatcag | 2460 |
| tgcccatgtg atggaagaag cccgtgcagg taaaaaaagt gtggcggatt tgatgcaaga | 2520 |
| aggcaggaca cttcttaaag ctgatgatgt catgcccggt gtagcccata tgatccacga | 2580 |
| agtggggatt gaagctaact tccctgatgg gacaaaactg gtaaccatcc atacccccgt | 2640 |
| tgaagatggt gggcataaat tggctccggg tgaagtgatt ttgaaaaacg aagacatcac | 2700 |
| tttgaatgca ggcaaacaag ccaccacttt agaagtgcat aacaaaggcg atcgccccgt | 2760 |
| gcaagtgggc tcccacttcc acttctttga agtgaataag cttttggaat ttgatcgtga | 2820 |
| aaaagcctat ggcaaacgcc tagacattgc ttctggaacc gctgtgcgct ttgaacccgg | 2880 |
| tgagaaaaaa accgtggaat tgattcaaat tggcggtaac caacgcattt acggctttaa | 2940 |
| ctctcttgtg gatcgccaag ccgatactga tggcaaaaaa cttgctctca aacgcgccaa | 3000 |
| agaacatggc tttggtgttg tgaattgcgg ttgcgataaa aaataaggaa aggacaatcc | 3060 |
| gatgaaaaaa atctctcgaa aagaatatgt ttctatgtat ggacccacta cgggcgataa | 3120 |
| agtgagattg ggcgataccg acctgatctt agaagtcgaa catgactgca ccacttatgg | 3180 |
| cgaagaaatt aagtttggtg gcggtaaaac cattcgcgat gggatggcac aaaccaacag | 3240 |
| ccccagcagc cacgaactcg atcttgtgct cactaacgcc ctgatcgtgg attacaccgg | 3300 |
| catttataaa gccgatattg gcattaaaaa tggcaaaatc catggcattg gcaaagcagg | 3360 |
| caataaagac atgcaagatg gcgtttgcaa caatctttgc gtgggccctg ctactgaggc | 3420 |
| tttggccgct gaagggctga ttgttacagc tggtgggatt gacacccaca tccactttat | 3480 |
| ttctccccaa caaatcccca cagcatttgc cagcgggatc acaaccatga ttggtggggg | 3540 |
| aacaggtcca gctgatggga ctaacgcgac taccatcact ccggggcgct ggaaccttaa | 3600 |
| aaccatgctc cgtgcctctg aagaatatgc catgaacttg ggctatttgg gtaaagggaa | 3660 |
| tgtgtcttat gaaccctccc tggtcgatca actcgaagct ggagccattg gctttaaaat | 3720 |
| ccacgaagac tggggtagca cacctgcagc catctaccat tgcttgaatg tggctgacaa | 3780 |
| atacgatgtg caagtggcta tccacaccga taccttgaat gaagcgggct gtgtggaaga | 3840 |
| cactttgcaa gccattgctg ggcgcactat ccacactttc cacactgaag gtgctggtgg | 3900 |
| cgggcacgct ccggatgtca ttaagatgtc tggcgaattt aacatcctcc cagcttctac | 3960 |
| caaccccacc attcctttca ccgtgaatac agaagccgaa cacatggaca tgttgatggt | 4020 |
| gtgccaccac ttggataaaa acatcaaaga agatgtccag tttgctgatt ctaggattcg | 4080 |
| cccccaaacc atcgccgctg aggacaaact ccacgatatg gggattttct ctatcaccag | 4140 |
| ctctgactcc caagcgatgg gccgtgtagg cgaggtcatc acccgcactt ggcaaacagc | 4200 |
| ggacaaaaac aaaaaagaat ttggtcgctt gcctgaggaa aaaggcgata atgacaactt | 4260 |
| ccgcatcaag cgctacattt ccaaatacac catcaacccc gctattgcac acggcatttc | 4320 |
| tgaatatgtc ggctctgtag aagtgggcaa attcgccgat ttggtgcttt ggagtcctgc | 4380 |
| gttctttggc attaaaccca acatgatcat caaaggcgga ttcatcgcac tttctcaaat | 4440 |
| gggcgatgcc aatgcctcta tccccactcc ccaacccgtg tattaccgcg aaatgtttgg | 4500 |
| ccaccatggt aaagccaaat ttgacaccaa tatcactttt gtatcccaag tggcttatga | 4560 |
| caacggcatt aaagaagagt tgggcttgca aagagtggtt ttgccagtta aaaactgccg | 4620 |
| caacatcacc aaaaaagacc tcaaattcaa cgatgttacc gcacacatcg aagtcaatcc | 4680 |
| tgaaacctac aaagttaaag tggatggcaa agaggttact tccaaagcag cggataaaat | 4740 |
| cagcctagca caactctaca acttgttcta ggtctttttc aaggagggat ggagggggtt | 4800 |
| ttctagtttt gatttttggc attgttgggg ggagtcgatt tactttattg gtttataatg | 4860 |
| aagtcaagag aatttttttc gccacagccc acagcacttg ttttggggtt tttgcgtgcg | 4920 |
| tgatggaggc gataggaaac tctaaatttt caccaaagga tggtcatgtt aggacttgtg | 4980 |
| ctgttgtatg ttgcgatcgt tttgattagc aacggggttt gcggtcttgc caatgtcgat | 5040 |
| gcaagaagca aggctgtgat gaatgtgttt gtaggtgggc tttccatcat ctgtaatgtg | 5100 |
| attgccatcg tctattccac tttcaacccc acgcctacag taacaggtgc agaggatgtc | 5160 |
| gctcaggtgt ctcaacactt gattaacttc tatggacctg ctacgggctt gttatttggt | 5220 |
| tttacctata tgtatgcggc gatcaacaac atctataatt tggattggaa accttatggg | 5280 |
| tggtattgtt tgtttgttac catcaacacc atcccagcgg cgatcctttc tcactactca | 5340 |
| gatgcacttg atgatcaccg ccttttaggc atcactgagg gtgattggtg ggcgtttatt | 5400 |
| tggctcgctt ggggcgtgtt gtggctcact ggttggattg aatgtgcgct aggcaaaagc | 5460 |
| ttgggcaaat ttgtcccatg gcttgccatc atcgaaggtg tgatcactgc ttggatccct | 5520 |
| gcttggttgc tcttcatcca acactggtca tgaggctttg aacttgcttg cagaaagcgt | 5580 |
| tctagggaat ttaaaggagg ggggtagctc taaggagctg gattttattg atttggagtg | 5640 |
| gtttgatgcc caaaaaagaa tggggcgttt cacttcacaa aaaggtgcgg agttggttct | 5700 |
| taagctcaaa aaccccccca agatgggctt atgcgatggg gatattctct ttgaggatgc | 5760 |
| cacaagccta attgctatca acatcatccc cacgcccacc ctccatgtct atgctgatag | 5820 |
| cacggcgcag gtggcgcgtt tgtgctatga agtggggaat cgccacgctt cgctttacta | 5880 |
| tggggatagc cctttgagtt ttaaaacccc atttgagaga cccttacaag tcttgtttga | 5940 |
| caaattagcc ttgcgctatg aggttttaaa aagcaaattg gatgcctcgc aacgcattag | 6000 |
| tgtgagcgca ccccatgccg atcctctgca ggagggtagt gcgcctttga agtttaaaag | 6060 |
| tgctcttgat ttgcaaattg tgataaaaaa gtaggtctat gcaaaaagat tttttattgc | 6120 |
| tccaagtgaa cgatgccatg ttccccattg ggagttacac ccactccttt gggttagaaa | 6180 |
| cctatatcca gcacaaggaa gtttccagca aagagagtgc gcttgattac atgcgcgcct | 6240 |
| acctctctac ccagttttta tacactgagc tcttagcgct taagttggct tatggctatg | 6300 |
| cccatcaaga ggacctagag tccattctag agatggagga acagatttgt ttagccaccc | 6360 |
| cgcctttaga attgcgtagc gccaaccaaa aattggggaa tcgcttttta aagaccattg | 6420 |
| gcgtgttgga tctccccttg agcgcctttt ttaaagacta catgcaaaag agcaccaccc | 6480 |
| ccacgcatgc cagtgcttat ggggtctttt gtgcatgctt gggattggga ttagaggcta | 6540 |
| gcctcaaaca ctatctctac gcgcaaagct ctaacatggt gattaattgt gtcaaaacca | 6600 |
| tccccctagc ccaaaatgac gggcaacgca ttttattagc cttgcaagag acttttacaa | 6660 |
| ccttgctcgt cactttagag ggcttggacg caagctatct gtgtgcagcc agtatccaaa | 6720 |
| atgacatcaa ggcgatgcaa cacgagcatt tatactccag actttatatg tcttaatcca | 6780 |
| tttagcaaag gaaaatcatg gttaaaatcg gtgtttgtgg tcctgtgggc agtggcaaaa | 6840 |
| cggctttgat cgaggccctg acaagggcga tgagccaaca atacagcata ggtgtcatca | 6900 |
| ctaatgatat ttatactaag gaagatgcgg agtttttgtg tagaaactct gtgatgccta | 6960 |
| gagagaggat cattggggta gagacggggg gttgcccgca caccgccatt agggaggacg | 7020 |
| cgtccatgaa tttggaagcg gtggaagaat tgcatgccaa attccccgat attgaattga | 7080 |
| tgtttattga gagtgggggg gataatctct ccaccacctt taatcccgag ctagccgatt | 7140 |
| ttacgatctt tgtgatcgat gtagcagagg gggataaaat cccacgcaag gggggtccgg | 7200 |
| gcatcacccg ctccgatctg ctcatcatca ataaaattga tcttgccccc tatgtgggcg | 7260 |
| cagatttggg cgtgatggat cgcgattcta agaaaatgcg cggggataaa ccctttttat | 7320 |
| tcaccaatat ccgctccaaa gagggcttag atcatgtgat tggctggatc aaaaaatacg | 7380 |
| ccttgctcga ggattaaact tggcagtgca acaccccacc agctacgccc aagcttttag | 7440 |
| gctttatttg aggaccaaaa tcgggcaaaa tggcaagtgc gtgatcgccg ataactactt | 7500 |
| tagcccaccc tttaagctca tgcccccttt ctatgaaaga ggcaatatgg ctgagatcat | 7560 |
| cttgatcgct gtgagcccgg ggatgctcaa aggcgatgcc caagagattc aaatggacat | 7620 |
| tggggaaaat tgccagcttg agctctcttt tcaaagtttt gagaaaatcc aagatacaga | 7680 |
| ggatggttac gcaacgcgta acacccaaat ccatatccac ccaaatgctt tgctggactt | 7740 |
| ctcccccctg cccatcatcc cctttgccaa cgcacacttt aaaaaccata gccatattgt | 7800 |
| gttgcacaaa aacgcccaac tgctctatag cgaaatcatc accgccgggc gcattgcgat | 7860 |
| gggcgaggct tttgccttcc ataaaatcca ctccacgctc aaaatttcca cccaagaggg | 7920 |
| cgagtctctt agaagtgttt ttttagataa cacgatttta gagcctgcca gcatggattt | 7980 |
| gaaaaaccca tgcatgtttg gtaactacac gcattatttg aatgggattt ttttaacacg | 8040 |
| cctattaaaa ttagaggatc ttttagaatt gttggagagc acacagatca acgctggggt | 8100 |
| gagcctcttg cctactagat tgccagaggg ctatcatagc ctatgtttaa aagccctagc | 8160 |
| caacggctca gagcctttat tagccttgcg taagagcatt tcaaacttgc tggtggaacg | 8220 |
| catggagggt tgagcaggtt agggtgctgg gtgtcaaagc cccagtatct agactcaaaa | 8280 |
| tcctctttcc caaacagccc atcaaggcta gccacccaag ccccatattt agattctttg | 8340 |
| tgttagcatg tagcattcaa ggctttaata aaggatgacc atggcttata gcattagcaa | 8400 |
| gaaaatt | 8407 |

The present invention also relates to an isolated nucleic acid molecule from a Helicobacter bizzozeronii urease gene cluster. This nucleic acid molecule is one of the following genes located in SEQ ID NO: 1: ureA; ureB; ureE; ureF; ureG; ureH; and ureI.

The present invention also relates to an isolated ureA gene of H. bizzozeronii, which has a nucleotide sequence corresponding to SEQ ID NO: 2 as follows:

| | |
|---|---|
| atgaaattaa cccctaaaga gctggacaag ctcatgttgc attatgcggg cgaattggct | 60 |
| aaaaaacgca aagcaaatgg cgttaagcta aattatactg aggcagtagc cctcatcagt | 120 |
| gcccatgtga tggaagaagc ccgtgcaggt aaaaaaagtg tggcggattt gatgcaagaa | 180 |
| ggcaggacac ttcttaaagc tgatgatgtc atgcccggtg tagcccatat gatccacgaa | 240 |
| gtggggattg aagctaactt ccctgatggg acaaaactgg taaccatcca tacccccgtt | 300 |
| gaagatggtg ggcataaatt ggctccgggt gaagtgattt tgaaaaacga agacatcact | 360 |
| ttgaatgcag gcaaacaagc caccacttta gaagtgcata acaaaggcga tcgccccgtg | 420 |
| caagtgggct cccacttcca cttctttgaa gtgaataagc ttttggaatt tgatcgtgaa | 480 |
| aaagcctatg gcaaacgcct agacattgct tctggaaccg ctgtgcgctt tgaacccggt | 540 |
| gagaaaaaaa ccgtggaatt gattcaaatt ggcggtaacc aacgcattta cggctttaac | 600 |
| tctcttgtgg atcgccaagc cgatactgat ggcaaaaaac ttgctctcaa acgcgccaaa | 660 |
| gaacatggct ttggtgttgt gaattgcggt tgcgataaaa aataa | 705 |

The nucleic acid molecule of the present invention encoding the ureA gene (SEQ ID NO: 2) of H. bizzozeronii is located in an open reading frame of SEQ ID NO: 1, which open reading frame extends from position 2,342 to position 3,046 of SEQ ID NO: 1.

The present invention also relates to a UreA protein or polypeptide, encoded by the nucleotide sequence corresponding to SEQ ID NO: 2, where the encoded protein or polypeptide has an amino acid sequence corresponding to SEQ ID NO: 3 as follows:

```
Met Lys Leu Thr Pro Lys Glu Leu Asp Lys Leu Met Leu His Tyr Ala
1 5 10 15
Gly Glu Leu Ala Lys Lys Arg Lys Ala Asn Gly Val Lys Leu Asn Tyr
                        20 25 30
Thr Glu Ala Val Ala Leu Ile Ser Ala His Val Met Glu Glu Ala Arg
                35 40 45
Ala Gly Lys Lys Ser Val Ala Asp Leu Met Gln Glu Gly Arg Thr Leu
        50 55 60
Leu Lys Ala Asp Asp Val Met Pro Gly Val Ala His Met Ile His Glu
65 70 75 80
Val Gly Ile Glu Ala Asn Phe Pro Asp Gly Thr Lys Leu Val Thr Ile
                                85 90 95
His Thr Pro Val Glu Asp Gly Gly His Lys Leu Ala Pro Gly Glu Val
                        100 105 110
Ile Leu Lys Asn Glu Asp Ile Thr Leu Asn Ala Gly Lys Gln Ala Thr
                115 120 125
Thr Leu Glu Val His Asn Lys Gly Asp Arg Pro Val Gln Val Gly Ser
        130 135 140
His Phe His Phe Phe Glu Val Asn Lys Leu Leu Glu Phe Asp Arg Glu
145 150 155 160
Lys Ala Tyr Gly Lys Arg Leu Asp Ile Ala Ser Gly Thr Ala Val Arg
                                165 170 175
Phe Glu Pro Gly Glu Lys Lys Thr Val Glu Leu Ile Gln Ile Gly Gly
                        180 185 190
Asn Gln Arg Ile Tyr Gly Phe Asn Ser Leu Val Asp Arg Gln Ala Asp
                195 200 205
Thr Asp Gly Lys Lys Leu Ala Leu Lys Arg Ala Lys Glu His Gly Phe
        210 215 220
Gly Val Val Asn Cys Gly Cys Asp Lys Lys
225 230
```

This protein or polypeptide has an estimated molecular weight of approximately 26 to 27 kilodaltons based on the deduced amino acid sequence.

The present invention also relates to an isolated ureB gene of H. bizzozeronii, which has a nucleotide sequence corresponding to SEQ ID NO: 4 as follows:

| | |
|---|---|
| atgaaaaaaa tctctcgaaa agaatatgtt tctatgtatg gacccactac gggcgataaa | 60 |
| gtgagattgg gcgataccga cctgatctta gaagtcgaac atgactgcac cacttatggc | 120 |
| gaagaaatta agtttggtgg cggtaaaacc attcgcgatg ggatggcaca aaccaacagc | 180 |
| cccagcagcc acgaactcga tcttgtgctc actaacgccc tgatcgtgga ttacaccggc | 240 |
| atttataaag ccgatattgg cattaaaaat ggcaaaatcc atggcattgg caaagcaggc | 300 |
| aataaagaca tgcaagatgg cgtttgcaac aatctttgcg tgggccctgc tactgaggct | 360 |
| ttggccgctg aagggctgat tgttacagct ggtgggattg acacccacat ccactttatt | 420 |
| tctccccaac aaatccccac agcatttgcc agcgggatca caaccatgat tggtggggga | 480 |
| acaggtccag ctgatgggac taacgcgact accatcactc cggggcgctg gaaccttaaa | 540 |
| accatgctcc gtgcctctga agaatatgcc atgaacttgg gctatttggg taaagggaat | 600 |
| gtgtcttatg aaccctccct ggtcgatcaa ctcgaagctg gagccattgg ctttaaaatc | 660 |
| cacgaagact ggggtagcac acctgcagcc atctaccatt gcttgaatgt ggctgacaaa | 720 |
| tacgatgtgc aagtggctat ccacaccgat accttgaatg aagcgggctg tgtggaagac | 780 |
| actttgcaag ccattgctgg gcgcactatc cacactttcc acactgaagg tgctggtggc | 840 |
| gggcacgctc cggatgtcat taagatgtct ggcgaattta acatcctccc agcttctacc | 900 |
| aaccccacca ttcctttcac cgtgaataca gaagccgaac acatggacat gttgatggtg | 960 |
| tgccaccact tggataaaaa catcaaagaa gatgtccagt ttgctgattc taggattcgc | 1020 |
| ccccaaacca tcgccgctga ggacaaactc cacgatatgg ggattttctc tatcaccagc | 1080 |
| tctgactccc aagcgatggg ccgtgtaggc gaggtcatca cccgcacttg gcaaacagcg | 1140 |
| gacaaaaaca aaaaagaatt tggtcgcttg cctgaggaaa aaggcgataa tgacaacttc | 1200 |
| cgcatcaagc gctacatttc caaatacacc atcaaccccg ctattgcaca cggcatttct | 1260 |
| gaatatgtcg gctctgtaga agtgggcaaa ttcgccgatt tggtgctttg gagtcctgcg | 1320 |
| ttctttggca ttaaacccaa catgatcatc aaaggcggat tcatcgcact ttctcaaatg | 1380 |
| ggcgatgcca atgcctctat ccccactccc caacccgtgt attaccgcga aatgtttggc | 1440 |
| caccatggta aagccaaatt tgacaccaat atcacttttg tatcccaagt ggcttatgac | 1500 |
| aacggcatta aagaagagtt gggcttgcaa agagtggttt tgccagttaa aaactgccgc | 1560 |
| aacatcacca aaaaagacct caaattcaac gatgttaccg cacacatcga agtcaatcct | 1620 |
| gaaacctaca aagttaaagt ggatggcaaa gaggttactt ccaaagcagc ggataaaatc | 1680 |
| agcctagcac aactctacaa cttgttctag | 1710 |

The nucleic acid molecule of the present invention encoding the ureB gene (SEQ ID NO: 4) of H. bizzozeronii is located in an open reading frame of SEQ ID NO: 1, which open reading frame extends from position 3,062 to position 4,771 of SEQ ID NO: 1.

The present invention also relates to a UreB protein or polypeptide, encoded by the nucleotide sequence corresponding to SEQ ID NO: 4, where the encoded protein or polypeptide has an amino acid sequence corresponding to SEQ ID NO: 5 as follows:

```
Met Lys Lys Ile Ser Arg Lys Glu Tyr Val Ser Met Tyr Gly Pro Thr
1 5 10 15
Thr Gly Asp Lys Val Arg Leu Gly Asp Thr Asp Leu Ile Leu Glu Val
                        20 25 30
Glu His Asp Cys Thr Thr Tyr Gly Glu Glu Ile Lys Phe Gly Gly Gly
                35 40 45
Lys Thr Ile Arg Asp Gly Met Ala Gln Thr Asn Ser Pro Ser Ser His
        50 55 60
Glu Leu Asp Leu Val Leu Thr Asn Ala Leu Ile Val Asp Tyr Thr Gly
65 70 75 80
Ile Tyr Lys Ala Asp Ile Gly Ile Lys Asn Gly Lys Ile His Gly Ile
                                85 90 95
Gly Lys Ala Gly Asn Lys Asp Met Gln Asp Gly Val Cys Asn Asn Leu
                        100 105 110
Cys Val Gly Pro Ala Thr Glu Ala Leu Ala Ala Glu Gly Leu Ile Val
                115 120 125
Thr Ala Gly Gly Ile Asp Thr His Ile His Phe Ile Ser Pro Gln Gln
        130 135 140
Ile Pro Thr Ala Phe Ala Ser Gly Ile Thr Thr Met Ile Gly Gly Gly
145 150 155 160
Thr Gly Pro Ala Asp Gly Thr Asn Ala Thr Thr Ile Thr Pro Gly Arg
                                165 170 175
Trp Asn Leu Lys Thr Met Leu Arg Ala Ser Glu Glu Tyr Ala Met Asn
                        180 185 190
Leu Gly Tyr Leu Gly Lys Gly Asn Val Ser Tyr Glu Pro Ser Leu Val
                195 200 205
Asp Gln Leu Glu Ala Gly Ala Ile Gly Phe Lys Ile His Glu Asp Trp
        210 215 220
Gly Ser Thr Pro Ala Ala Ile Tyr His Cys Leu Asn Val Ala Asp Lys
225 230 235 240
Tyr Asp Val Gln Val Ala Ile His Thr Asp Thr Leu Asn Glu Ala Gly
                                245 250 255
Cys Val Glu Asp Thr Leu Gln Ala Ile Ala Gly Arg Thr Ile His Thr
                        260 265 270
Phe His Thr Glu Gly Ala Gly Gly Gly His Ala Pro Asp Val Ile Lys
                275 280 285
Met Ser Gly Glu Phe Asn Ile Leu Pro Ala Ser Thr Asn Pro Thr Ile
        290 295 300
Pro Phe Thr Val Asn Thr Glu Ala Glu His Met Asp Met Leu Met Val
305 310 315 320
Cys His His Leu Asp Lys Asn Ile Lys Glu Asp Val Gln Phe Ala Asp
                                325 330 335
Ser Arg Ile Arg Pro Gln Thr Ile Ala Ala Glu Asp Lys Leu His Asp
                        340 345 350
Met Gly Ile Phe Ser Ile Thr Ser Ser Asp Ser Gln Ala Met Gly Arg
                355 360 365
Val Gly Glu Val Ile Thr Arg Thr Trp Gln Thr Ala Asp Lys Asn Lys
        370 375 380
Lys Glu Phe Gly Arg Leu Pro Glu Glu Lys Gly Asp Asn Asp Asn Phe
385 390 395 400
Arg Ile Lys Arg Tyr Ile Ser Lys Tyr Thr Ile Asn Pro Ala Ile Ala
                                405 410 415
His Gly Ile Ser Glu Tyr Val Gly Ser Val Glu Val Gly Lys Phe Ala
                        420 425 430
Asp Leu Val Leu Trp Ser Pro Ala Phe Phe Gly Ile Lys Pro Asn Met
                435 440 445
Ile Ile Lys Gly Gly Phe Ile Ala Leu Ser Gln Met Gly Asp Ala Asn
        450 455 460
Ala Ser Ile Pro Thr Pro Gln Pro Val Tyr Tyr Arg Glu Met Phe Gly
465 470 475 480
His His Gly Lys Ala Lys Phe Asp Thr Asn Ile Thr Phe Val Ser Gln
                                485 490 495
Val Ala Tyr Asp Asn Gly Ile Lys Glu Glu Leu Gly Leu Gln Arg Val
                        500 505 510
Val Leu Pro Val Lys Asn Cys Arg Asn Ile Thr Lys Lys Asp Leu Lys
                515 520 525
Phe Asn Asp Val Thr Ala His Ile Glu Val Asn Pro Glu Thr Tyr Lys
        530 535 540
Val Lys Val Asp Gly Lys Glu Val Thr Ser Lys Ala Ala Asp Lys Ile
545 550 555 560
Ser Leu Ala Gln Leu Tyr Asn Leu Phe
                                565
```

This protein or polypeptide has an estimated molecular weight of approximately 60 kilodaltons based on the deduced amino acid sequence.

The present invention also relates to an isolated ureE gene of H. bizzozeronii, which has a nucleotide sequence corresponding to SEQ ID NO: 6 as follows:

| | |
|---|---|
| ttgcttgcag aaagcgttct agggaattta aaggaggggg gtagctctaa ggagctggat | 60 |
| tttattgatt tggagtggtt tgatgcccaa aaaagaatgg ggcgtttcac ttcacaaaaa | 120 |
| ggtgcggagt tggttcttaa gctcaaaaac ccccccaaga tgggcttatg cgatggggat | 180 |
| attctctttg aggatgccac aagcctaatt gctatcaaca tcatccccac gcccaccctc | 240 |
| catgtctatg ctgatagcac ggcgcaggtg gcgcgtttgt gctatgaagt ggggaatcgc | 300 |
| cacgcttcgc tttactatgg ggatagccct ttgagtttta aaaccccatt tgagagaccc | 360 |
| ttacaagtct tgtttgacaa attagccttg cgctatgagg ttttaaaaag caaattggat | 420 |
| gcctcgcaac gcattagtgt gagcgcaccc catgccgatc ctctgcagga gggtagtgcg | 480 |
| cctttgaagt ttaaaagtgc tcttgatttg caaattgtga taaaaaagta g | 531 |

The ureE gene (SEQ ID NO: 6) of H. bizzozeronii is located in an open reading frame of SEQ ID NO: 1, which open reading frame extends from position 5,564 to position 6,094 of SEQ ID NO: 1.

The present invention also relates to a UreE protein or polypeptide, encoded by the nucleotide sequence corresponding to SEQ ID NO: 6, where the encoded protein or polypeptide has an amino acid sequence corresponding to SEQ ID NO: 7 as follows:

```
Met Leu Ala Glu Ser Val Leu Gly Asn Leu Lys Glu Gly Gly Ser Ser
1 5 10 15
Lys Glu Leu Asp Phe Ile Asp Leu Glu Trp Phe Asp Ala Gln Lys Arg
                        20 25 30
Met Gly Arg Phe Thr Ser Gln Lys Gly Ala Glu Leu Val Leu Lys Leu
                35 40 45
Lys Asn Pro Pro Lys Met Gly Leu Cys Asp Gly Asp Ile Leu Phe Glu
        50 55 60
Asp Ala Thr Ser Leu Ile Ala Ile Asn Ile Ile Pro Thr Pro Thr Leu
65 70 75 80
His Val Tyr Ala Asp Ser Thr Ala Gln Val Ala Arg Leu Cys Tyr Glu
                                85 90 95
Val Gly Asn Arg His Ala Ser Leu Tyr Tyr Gly Asp Ser Pro Leu Ser
                        100 105 110
Phe Lys Thr Pro Phe Glu Arg Pro Leu Gln Val Leu Phe Asp Lys Leu
                115 120 125
Ala Leu Arg Tyr Glu Val Leu Lys Ser Lys Leu Asp Ala Ser Gln Arg
        130 135 140
Ile Ser Val Ser Ala Pro His Ala Asp Pro Leu Gln Glu Gly Ser Ala
145 150 155 160
Pro Leu Lys Phe Lys Ser Ala Leu Asp Leu Gln Ile Val Ile Lys Lys
                                165 170 175
```

This protein or polypeptide has an estimated molecular weight of approximately 19 to 20 kilodaltons based on the deduced amino acid sequence.

The present invention also relates to an isolated ureF gene of H. bizzozeronii, which has a nucleotide sequence corresponding to SEQ ID NO: 8 as follows:

| | |
|---|---|
| atgcaaaaag attttttatt gctccaagtg aacgatgcca tgttccccat tgggagttac | 60 |
| acccactcct ttgggttaga aacctatatc cagcacaagg aagtttccag caaagagagt | 120 |
| gcgcttgatt acatgcgcgc ctacctctct acccagtttt tatacactga gctcttagcg | 180 |
| cttaagttgg cttatggcta tgcccatcaa gaggacctag agtccattct agagatggag | 240 |
| gaacagattt gtttagccac cccgccttta gaattgcgta gcgccaacca aaaattgggg | 300 |
| aatcgctttt taaagaccat tggcgtgttg gatctcccct tgagcgcctt ttttaaagac | 360 |
| tacatgcaaa agagcaccac ccccacgcat gccagtgctt atggggtctt ttgtgcatgc | 420 |
| ttgggattgg gattagaggc tagcctcaaa cactatctct acgcgcaaag ctctaacatg | 480 |
| gtgattaatt gtgtcaaaac catcccccta gcccaaaatg acgggcaacg cattttatta | 540 |
| gccttgcaag agacttttac aaccttgctc gtcactttag agggcttgga cgcaagctat | 600 |
| ctgtgtgcag ccagtatcca aaatgacatc aaggcgatgc aacacgagca tttatactcc | 660 |
| agactttata tgtcttaa | 678 |

The ureF gene (SEQ ID NO: 8) of H. bizzozeronii is located in an open reading frame of SEQ ID NO: 1, which open reading frame extends from position 6,099 to position 6,776 of SEQ ID NO: 1.

The present invention also relates to a UreF protein or polypeptide, encoded by the nucleotide sequence corresponding to SEQ ID NO: 8, where the encoded protein or polypeptide has an amino acid sequence corresponding to SEQ ID NO: 9 as follows:

```
Met Gln Lys Asp Phe Leu Leu Leu Gln Val Asn Asp Ala Met Phe Pro
1 5 10 15
Ile Gly Ser Tyr Thr His Ser Phe Gly Leu Glu Thr Tyr Ile Gln His
                        20 25 30
Lys Glu Val Ser Ser Lys Glu Ser Ala Leu Asp Tyr Met Arg Ala Tyr
                35 40 45
Leu Ser Thr Gln Phe Leu Tyr Thr Glu Leu Leu Ala Leu Lys Leu Ala
        50 55 60
Tyr Gly Tyr Ala His Gln Gln Asp Leu Gln Ser Ile Leu Glu Met Gln
65 70 75 80
Glu Gln Ile Cys Leu Ala Thr Pro Pro Leu Gln Leu Arg Ser Ala Asn
                                85 90 95
Gln Lys Leu Gly Asn Arg Phe Leu Lys Thr Ile Gly Val Leu Asp Leu
                        100 105 110
Pro Leu Ser Ala Phe Phe Lys Asp Tyr Met Gln Lys Ser Thr Thr Pro
                115 120 125
Thr His Ala Ser Ala Tyr Gly Val Phe Cys Ala Cys Leu Gly Leu Gly
        130 135 140
Leu Glu Ala Ser Leu Lys His Tyr Leu Tyr Ala Gln Ser Ser Asn Met
145 150 155 160
Val Ile Asn Cys Val Lys Thr Ile Pro Leu Ala Gln Asn Asp Gly Gln
                                165 170 175
Arg Ile Leu Leu Ala Leu Gln Glu Thr Phe Thr Thr Leu Leu Val Thr
                        180 185 190
Leu Gln Gly Leu Asp Ala Ser Tyr Leu Cys Ala Ala Ser Ile Gln Asn
                195 200 205
Asp Ile Lys Ala Met Gln His Glu His Leu Tyr Ser Arg Leu Tyr Met
        210 215 220
Ser
225
```

This protein or polypeptide has an estimated molecular weight of approximately 28 to 29 kilodaltons based on the deduced amino acid sequence.

The present invention also relates to an isolated ureG gene of H. bizzozeronii, which has a nucleotide sequence corresponding to SEQ ID NO: 10 as follows:

| | |
|---|---|
| atggttaaaa tcggtgtttg tggtcctgtg ggcagtggca aaacggcttt gatcgaggcc | 60 |
| ctgacaaggg cgatgagcca acaatacagc ataggtgtca tcactaatga tatttatact | 120 |
| aaggaagatg cggagttttt gtgtagaaac tctgtgatgc ctagagagag gatcattggg | 180 |
| gtagagacgg ggggttgccc gcacaccgcc attagggagg acgcgtccat gaatttggaa | 240 |
| gcggtggaag aattgcatgc caaattcccc gatattgaat tgatgtttat tgagagtggg | 300 |
| ggggataatc tctccaccac ctttaatccc gagctagccg attttacgat ctttgtgatc | 360 |
| gatgtagcag agggggataa aatcccacgc aaggggggtc cgggcatcac ccgctccgat | 420 |
| ctgctcatca tcaataaaat tgatcttgcc ccctatgtgg gcgcagattt gggcgtgatg | 480 |
| gatcgcgatt ctaagaaaat gcgcggggat aaaccctttt tattcaccaa tatccgctcc | 540 |
| aaagagggct tagatcatgt gattggctgg atcaaaaaat acgccttgct cgaggattaa | 600 |

The ureG gene (SEQ ID NO: 10) of H. bizzozeronii is located in an open reading frame of SEQ ID NO: 1, which open reading frame extends from position 6,798 to position 7,397 of SEQ ID NO: 1.

The present invention also relates to a UreG protein or polypeptide, encoded by the nucleotide sequence corresponding to SEQ ID NO: 10, where the encoded protein or polypeptide has an amino acid sequence corresponding to SEQ ID NO: 11 as follows:

```
Met Val Lys Ile Gly Val Cys Gly Pro Val Gly Ser Gly Lys Thr Ala
1 5 10 15
Leu Ile Glu Ala Leu Thr Arg Ala Met Ser Gln Gln Tyr Ser Ile Gly
                        20 25 30
Val Ile Thr Asn Asp Ile Tyr Thr Lys Glu Asp Ala Glu Phe Leu Cys
                35 40 45
Arg Asn Ser Val Met Pro Arg Glu Arg Ile Ile Gly Val Glu Thr Gly
        50 55 60
Gly Cys Pro His Thr Ala Ile Arg Glu Asp Ala Ser Met Asn Leu Glu
65 70 75 80
Ala Val Glu Glu Leu His Ala Lys Phe Pro Asp Ile Glu Leu Met Phe
                                85 90 95
Ile Glu Ser Gly Gly Asp Asn Leu Ser Thr Thr Phe Asn Pro Glu Leu
                        100 105 110
Ala Asp Phe Thr Ile Phe Val Ile Asp Val Ala Glu Gly Asp Lys Ile
                115 120 125
Pro Arg Lys Gly Gly Pro Gly Ile Thr Arg Ser Asp Leu Leu Ile Ile
        130 135 140
Asn Lys Ile Asp Leu Ala Pro Tyr Val Gly Ala Asp Leu Gly Val Met
145 150 155 160
Asp Arg Asp Ser Lys Lys Met Arg Gly Asp Lys Pro Phe Leu Phe Thr
                                165 170 175
Asn Ile Arg Ser Lys Glu Gly Leu Asp His Val Ile Gly Trp Ile Lys
                        180 185 190
Lys Tyr Ala Leu Leu Glu Asp
                195
```

This protein or polypeptide has an estimated molecular weight of approximately 21 to 22 kilodaltons based on the deduced amino acid sequence.

The present invention also relates to an isolated nucleic acid molecule encoding a ureH gene of H. bizzozeronii, which has a nucleotide sequence corresponding to SEQ ID NO: 12 as follows:

| | |
|---|---|
| ttggcagtgc aacaccccac cagctacgcc caagctttta ggctttattt gaggaccaaa | 60 |
| atcgggcaaa atggcaagtg cgtgatcgcc gataactact ttagcccacc ctttaagctc | 120 |
| atgccccctt tctatgaaag aggcaatatg gctgagatca tcttgatcgc tgtgagcccg | 180 |
| gggatgctca aaggcgatgc ccaagagatt caaatggaca ttggggaaaa ttgccagctt | 240 |
| gagctctctt ttcaaagttt tgagaaaatc caagatacag aggatggtta cgcaacgcgt | 300 |
| aacacccaaa tccatatcca cccaaatgct ttgctggact tctcccccct gcccatcatc | 360 |
| ccctttgcca acgcacactt taaaaaccat agccatattg tgttgcacaa aaacgcccaa | 420 |
| ctgctctata gcgaaatcat caccgccggg cgcattgcga tgggcgaggc ttttgccttc | 480 |
| cataaaatcc actccacgct caaaatttcc acccaagagg gcgagtctct tagaagtgtt | 540 |
| tttttagata acacgatttt agagcctgcc agcatggatt tgaaaaaccc atgcatgttt | 600 |
| ggtaactaca cgcattattt gaatgggatt tttttaacac gcctattaaa attagaggat | 660 |
| cttttagaat tgttggagag cacacagatc aacgctgggg tgagcctctt gcctactaga | 720 |
| ttgccagagg gctatcatag cctatgttta aaagccctag ccaacggctc agagccttta | 780 |
| ttagccttgc gtaagagcat ttcaaacttg ctggtggaac gcatggaggg ttga | 834 |

The ureH gene (SEQ ID NO: 12) of H. bizzozeronii is located in an open reading frame of SEQ ID NO: 1, which open reading frame extends from position 7,400 to position 8,233 of SEQ ID NO: 1.

The present invention also relates to a UreH protein or polypeptide, encoded by the nucleotide sequence corresponding to SEQ ID NO: 12, where the encoded protein or polypeptide has an amino acid sequence corresponding to SEQ ID NO: 13 as follows:

```
Met Ala Val Gln His Pro Thr Ser Tyr Ala Gln Ala Phe Arg Leu Tyr
1 5 10 15
Leu Arg Thr Lys Ile Gly Gln Asn Gly Lys Cys Val Ile Ala Asp Asn
                        20 25 30
Tyr Phe Ser Pro Pro Phe Lys Leu Met Pro Pro Phe Tyr Glu Arg Gly
                35 40 45
Asn Met Ala Glu Ile Ile Leu Ile Ala Val Ser Pro Gly Met Leu Lys
        50 55 60
Gly Asp Ala Gln Glu Ile Gln Met Asp Ile Gly Glu Asn Cys Gln Leu
65 70 75 80
Glu Leu Ser Phe Gln Ser Phe Glu Lys Ile Gln Asp Thr Glu Asp Gly
                                85 90 95
Tyr Ala Thr Arg Asn Thr Gln Ile His Ile His Pro Asn Ala Leu Leu
                        100 105 110
Asp Phe Ser Pro Leu Pro Ile Ile Pro Phe Ala Asn Ala His Phe Lys
                115 120 125
Asn His Ser His Ile Val Leu His Lys Asn Ala Gln Leu Leu Tyr Ser
        130 135 140
Glu Ile Ile Thr Ala Gly Arg Ile Ala Met Gly Glu Ala Phe Ala Phe
145 150 155 160
His Lys Ile His Ser Thr Leu Lys Ile Ser Thr Gln Glu Gly Glu Ser
                                165 170 175
Leu Arg Ser Val Phe Leu Asp Asn Thr Ile Leu Glu Pro Ala Ser Met
                        180 185 190
Asp Leu Lys Asn Pro Cys Met Phe Gly Asn Tyr Thr His Tyr Leu Asn
                195 200 205
Gly Ile Phe Leu Thr Arg Leu Leu Lys Leu Glu Asp Leu Leu Glu Leu
        210 215 220
Leu Glu Ser Thr Gln Ile Asn Ala Gly Val Ser Leu Leu Pro Thr Arg
225 230 235 240
Leu Pro Glu Gly Tyr His Ser Leu Cys Leu Lys Ala Leu Ala Asn Gly
                                245 250 255
Ser Glu Pro Leu Leu Ala Leu Arg Lys Ser Ile Ser Asn Leu Leu Val
                        260 265 270
Glu Arg Met Glu Gly
                275
```

This protein or polypeptide has an estimated molecular weight of approximately 29 to 30 kilodaltons based on the deduced amino acid sequence.

The present invention also relates to an isolated ureI gene of H. bizzozeronii, which has a nucleotide sequence corresponding to SEQ ID NO: 14 as follows:

| | |
|---|---|
| atggtcatgt taggacttgt gctgttgtat gttgcgatcg ttttgattag caacggggtt | 60 |
| tgcggtcttg ccaatgtcga tgcaagaagc aaggctgtga tgaatgtgtt tgtaggtggg | 120 |
| ctttccatca tctgtaatgt gattgccatc gtctattcca ctttcaaccc cacgcctaca | 180 |
| gtaacaggtg cagaggatgt cgctcaggtg tctcaacact tgattaactt ctatggacct | 240 |
| gctacgggct tgttatttgg ttttacctat atgtatgcgg cgatcaacaa catctataat | 300 |
| ttggattgga aaccttatgg gtggtattgt ttgtttgtta ccatcaacac catcccagcg | 360 |
| gcgatccttt ctcactactc agatgcactt gatgatcacc gccttttagg catcactgag | 420 |
| ggtgattggt gggcgtttat ttggctcgct tggggcgtgt tgtggctcac tggttggatt | 480 |
| gaatgtgcgc taggcaaaag cttgggcaaa tttgtcccat ggcttgccat catcgaaggt | 540 |
| gtgatcactg cttggatccc tgcttggttg ctcttcatcc aacactggtc atga | 594 |

The ureI gene (SEQ ID NO: 14) of H. bizzozeronii is located in an open reading frame of SEQ ID NO: 1, which open reading frame extends from position 4,960 to position 5,553 of SEQ ID NO: 1.

The present invention also relates to a UreI protein or polypeptide, encoded by the nucleotide sequence corresponding to SEQ ID NO: 14, where the encoded protein or polypeptide has an amino acid sequence corresponding to SEQ ID NO: 15 as follows:

```
Met Leu Gly Leu Val Leu Leu Tyr Val Ala Ile Val Leu Ile Ser Asn
1 5 10 15
Gly Val Cys Gly Leu Ala Asn Val Asp Ala Arg Ser Lys Ala Val Met
                        20 25 30
Asn Val Phe Val Gly Gly Leu Ser Ile Ile Cys Asn Val Ile Ala Ile
                35 40 45
Val Tyr Ser Thr Phe Asn Pro Thr Pro Thr Val Thr Gly Ala Glu Asp
        50 55 60
Val Ala Gln Val Ser Gln His Leu Ile Asn Phe Tyr Gly Pro Ala Thr
65 70 75 80
Gly Leu Leu Phe Gly Phe Thr Tyr Met Tyr Ala Ala Ile Asn Asn Ile
                                85 90 95
Tyr Asn Leu Asp Trp Lys Pro Tyr Gly Trp Tyr Cys Leu Phe Val Thr
                        100 105 110
Ile Asn Thr Ile Pro Ala Ala Ile Leu Ser His Tyr Ser Asp Ala Leu
                115 120 125
Asp Asp His Arg Leu Leu Gly Ile Thr Glu Gly Asp Trp Trp Ala Phe
        130 135 140
Ile Trp Leu Ala Trp Gly Val Leu Trp Leu Thr Gly Trp Ile Glu Cys
145 150 155 160
Ala Leu Gly Lys Ser Leu Gly Lys Phe Val Pro Trp Leu Ala Ile Ile
                                165 170 175
Glu Gly Val Ile Thr Ala Trp Ile Pro Ala Trp Leu Leu Phe Ile Gln
                        180 185 190
His Trp Ser
                195
```

This protein or polypeptide encoded by this amino acid sequence has an estimated molecular weight of approximately 21 to 22 kilodaltons based on the deduced amino acid sequence.

Also suitable as an isolated nucleic acid molecule according to the present invention is an isolated nucleic acid molecule including at least 20 contiguous nucleic acid residues that hybridize to a nucleic acid having a nucleotide sequence of SEQ ID NOs: 2, 4, 6, 8, 10, 12, and 14, or the complements of SEQ ID NOs: 2, 4, 6, 8, 10, 12, and 14, under stringent conditions. Homologous nucleotide sequences can be detected by selectively hybridizing to each other. Selectively hybridizing is used herein to mean hybridization of DNA or RNA probes from one sequence to the “homologous” sequence under stringent conditions which are characterized by a hybridization buffer comprising 2×SSC, 0.1% SDS at 56° C. (Ausubel et al., eds., Current Protocols in Molecular Biology, Vol. I, New York: Greene Publishing Associates, Inc. and John Wiley & Sons, Inc., p. 2.10.3 (1989), which is hereby incorporated by reference in its entirety). Another example of suitable stringency conditions is when hybridization is carried out at 65° C. for 20 hours in a medium containing 1 M NaCl, 50 mM Tris-HCl, pH 7.4, 10 mM EDTA, 0.1% sodium dodecyl sulfate, 0.2% ficoll, 0.2% polyvinylpyrrolidone, 0.2% bovine serum albumin, 50 μg/ml E. coli DNA. In one embodiment, the present invention is directed to isolated nucleic acid molecules having nucleotide sequences containing at least 20 contiguous nucleic acid residues that hybridize to the nucleic acid molecules of the present invention, namely, SEQ ID NOs: 2, 4, 6, 8, 10, 12, and 14, under stringent conditions including 50 percent formamide at 42° C.

Fragments of the above proteins or polypeptides are encompassed by the present invention.

The proteins or polypeptides of the present invention are preferably produced in purified form by conventional techniques. To isolate the proteins or polypeptides, a protocol involving a host cell such as Escherchia coli may be used, in which protocol the E. coli host cell carrying a recombinant plasmid is propagated, homogenized, and the homogenate is centrifuged to remove bacterial debris. The supernatant is then subjected to sequential ammonium sulfate precipitation. The fraction containing the proteins or polypeptides of the present invention are subjected to gel filtration in an appropriately sized dextran or polyacrylamide column to separate the proteins or polypeptides. If necessary, the protein fraction may be further purified by HPLC.

Fragments of the proteins or polypeptides of the present invention can be produced by digestion of a full-length elicitor protein with proteolytic enzymes like chymotrypsin or Staphylococcus proteinase A, or trypsin. Different proteolytic enzymes are likely to cleave the proteins or polypeptides of the present invention at different sites based on the amino acid sequence of the proteins or polypeptides. Some of the fragments that result from proteolysis may be active elicitors of resistance.

In another approach, based on knowledge of the primary structure of the protein or polypeptide, fragments of the genes encoding the proteins or polypeptides of the present invention may be synthesized by using the PCR technique together with specific sets of primers chosen to represent particular portions of the protein or polypeptide of interest. These then would be cloned into an appropriate vector for expression of a truncated peptide or protein.

Chemical synthesis can also be used to make suitable fragments. Such a synthesis is carried out using known amino acid sequences for the protein or polypeptide being produced. Alternatively, subjecting a full length protein or polypeptide of the present invention to high temperatures and pressures will produce fragments. These fragments can then be separated by conventional procedures (e.g., chromatography, SDS-PAGE).

Variants may also (or alternatively) be made, for example, by the deletion or addition of amino acids that have minimal influence on the properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification, or identification of the polypeptide.

The protein or polypeptide of the present invention is preferably produced in purified form (preferably at least about 80%, more preferably 90%, pure) by conventional techniques. Typically, the protein or polypeptide of the present invention is secreted into the growth medium of Helicobacter cells or host cells which express a functional type III secretion system capable of secreting the protein or polypeptide of the present invention. Alternatively, the protein or polypeptide of the present invention is produced but not secreted into growth medium of recombinant host cells (e.g., Escherichia coli). In such cases, to isolate the protein, the host cell (e.g., E. coli) carrying a recombinant plasmid is propagated, lysed by sonication, heat, differential pressure, or chemical treatment, and the homogenate is centrifuged to remove bacterial debris. The supernatant is then subjected to sequential ammonium sulfate precipitation. The fraction containing the polypeptide or protein of the present invention is subjected to gel filtration in an appropriately sized dextran or polyacrylamide column to separate the proteins. If necessary, the protein fraction may be further purified by HPLC.

The DNA molecule encoding the proteins or polypeptides of the present invention can be incorporated in cells using conventional recombinant DNA technology. Generally, this involves inserting the DNA molecule into an expression system to which the DNA molecule is heterologous (i.e., not normally present). The heterologous DNA molecule is inserted into the expression system or vector in sense orientation and correct reading frame. The vector contains the necessary elements for the transcription and translation of the inserted protein-coding sequences. Thus, the present invention also relates to a DNA construct containing the nucleic acid of the present invention, which is operably linked to both a 5′ promoter and a 3′ regulatory region (i.e., transcription terminator) capable of affording transcription and expression of the encoded proteins or polypeptides of the present invention in host cells or host organisms.

The present invention also relates to an expression vector containing a DNA molecule encoding the proteins or polypeptides of the present invention. The nucleic acid molecules of the present invention may be inserted into any of the many available expression vectors using reagents that are well known in the art. In preparing a DNA vector for expression, the various DNA sequences may normally be inserted or substituted into a bacterial plasmid. Any convenient plasmid may be employed, which will be characterized by having a bacterial replication system, a marker which allows for selection in a bacterium, and generally one or more unique, conveniently located restriction sites. The selection of a vector will depend on the preferred transformation technique and target host for transformation.

Suitable vectors for practicing the present invention include, but are not limited to, the following viral vectors such as lambda vector system gt11, gtWES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK +/− or KS +/− (see “Stratagene Cloning Systems” Catalog (1993)), pQE, pIH821, pGEX, pET series (Studier et al, “Use of T7 RNA Polymerase to Direct Expression of Cloned Genes,” Methods in Enzymology. 185:60-89 (1990) which is hereby incorporated by reference in its entirety), and any derivatives thereof. Any appropriate vectors now known or later described for genetic transformation are suitable for use with the present invention. Recombinant molecules can be introduced into cells via transformation, particularly transduction, conjugation, mobilization, or electroporation. The DNA sequences are cloned into the vector using standard cloning procedures in the art, as described by Maniatis et al., Molecular Cloning: A Laboratory Manual, Cold Springs Harbor, N.Y.: Cold Springs Laboratory, (1982), which is hereby incorporated by reference in its entirety.

U.S. Pat. No. 4,237,224 issued to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

A variety of host-vector systems may be utilized to express the protein-encoding sequence(s). Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and messenger RNA (mRNA) translation).

Transcription of DNA is dependent upon the presence of a promotor which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology, 68:473 (1979), which is hereby incorporated by reference in its entirety.

Promoters vary in their “strength” (i.e. their ability to promote transcription). For the purposes of expressing a cloned gene, it is generally desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promotor, lac promotor, trp promotor, recA promotor, ribosomal RNA promotor, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promotor or other E. coli promoters produced by recombinant DNA or other synthetic DNA techniques may be used to provide for transcription of the inserted gene.

Bacterial host cell strains and expression vectors may be chosen which inhibit the action of the promotor unless specifically induced. In certain operations, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promotor, may also contain any combination of various “strong” transcription and/or translation initiation signals. For instance, efficient translation in E. coli requires an SD sequence about 7-9 bases 5′ to the initiation codon (“ATG”) to provide a ribosome binding site. Thus, any SD-ATG combination that can be utilized by host cell ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

In one aspect of the present invention, the nucleic acid molecule of the present invention is incorporated into an appropriate vector in the sense direction, such that the open reading frame is properly oriented for the expression of the encoded protein under control of a promoter of choice. This involves the inclusion of the appropriate regulatory elements into the DNA-vector construct. These include non-translated regions of the vector, useful promoters, and 5′ and 3′ untranslated regions which interact with host cellular proteins to carry out transcription and translation. Such elements may vary in their strength and specificity. Depending on the vector system and host utilized, any number of suitable transcription and translation elements, including constitutive and inducible promoters, may be used.

A constitutive promoter is a promoter that directs expression of a gene throughout the development and life of an organism.

An inducible promoter is a promoter that is capable of directly or indirectly activating transcription of one or more DNA sequences or genes in response to an inducer. In the absence of an inducer, the DNA sequences or genes will not be transcribed.

The DNA construct of the present invention also includes an operable 3′ regulatory region, selected from among those which are capable of providing correct transcription termination and polyadenylation of mRNA for expression in the host cell of choice, operably linked to a DNA molecule which encodes for a protein of choice.

The vector of choice, promoter, and an appropriate 3′ regulatory region can be ligated together to produce the DNA construct of the present invention using well known molecular cloning techniques as described in Sambrook et al., Molecular Cloning: A Laboratory Manual, Second Edition, Cold Spring Harbor Press, NY (1989), and Ausubel, F. M. et al. Current Protocols in Molecular Biology, New York, N.Y: John Wiley & Sons, (1989), which are hereby incorporated by reference in their entirety.

Once the DNA construct of the present invention has been prepared, it is ready to be incorporated into a host cell. Accordingly, another aspect of the present invention relates to a method of making a recombinant cell. Basically, this method is carried out by transforming a host cell with a DNA construct of the present invention under conditions effective to yield transcription of the DNA molecule in the host cell. Recombinant molecules can be introduced into cells via transformation, particularly transduction, conjugation, mobilization, or electroporation. The DNA sequences are cloned into the host cell using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference in its entirety. Suitable host cells include, but are not limited to, bacteria, virus, yeast, mammalian cells, insect, plant, and the like.

Generally, the mammalian immune system responds to infection by pathogenic bacteria by producing antibodies that bind to specific proteins or carbohydrates on the bacterial surface. The antibodies stimulate binding to macrophages which have receptors that bind to the F<c> region of the antibodies. Other serum proteins, called complement, coat the foreign particle and stimulate their ingestion by binding to specific surface receptors on the macrophage. Once the particle is bound to the surface of the macrophage, the sequential process of ingestion begins by continual apposition of a segment of the plasma membrane to the particle surface. Surface receptors on the membranes then interact with ligands distributed uniformity over the particle surface to link the surfaces together. The macrophage enveloping the particle is then delivered to lysosomes where the particle is ingested.

In view of the present invention's determination of nucleotide sequences conferring on Helicobacter bizzozeronii an ability to produce functional urease, a wide array of therapeutic and/or prophylatic agents and diagnostic procedures for, respectively, treating and detecting Helicobacter bizzozeronii can be developed.

For example, an effective amount of the proteins or polypeptides of the present invention can be administered alone or in combination with a pharmaceutically-acceptable carrier to mammals such as canines and felines, as a vaccine, for preventing onset of disease resulting from infection by Helicobacter bizzozeronii. Alternatively, it is possible to administer to individuals exposed to Helicobacter bizzozeronii an effective amount of an antibody or binding portion thereof against these proteins or polypeptides as a passive immunization. Such antibodies or binding portions thereof are administered alone or in combination with a pharmaceutically-acceptable carrier to effect short term treatment of individuals who may have been recently exposed to Helicobacter bizzozeronii.

Antibodies suitable for use in inducing passive immunity can be monoclonal or polyclonal.

Monoclonal antibody production may be effected by techniques which are well-known in the art. Basically, the process involves first obtaining immune cells (lymphocytes) from the spleen of a mammal (e.g., mouse) which has been previously immunized with the antigen of interest (i.e., the protein or peptide of the present invention) either in vivo or in vitro. The antibody-secreting lymphocytes are then fused with (mouse) myeloma cells or transformed cells, which are capable of replicating indefinitely in cell culture, thereby producing an immortal, immunoglobulin-secreting cell line. The resulting fused cells, or hybridomas, are cultured and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975), which is hereby incorporated by reference in its entirety.

Mammalian lymphocytes are immunized by in vivo immunization of the animal (e.g., a mouse) with one of the proteins or polypeptides of the present invention. Such immunizations are repeated as necessary at intervals of up to several weeks to obtain a sufficient titer of antibodies. The virus is carried in appropriate solutions or adjuvants. Following the last antigen boost, the animals are sacrificed and spleen cells removed.

Fusion with mammalian myeloma cells or other fusion partners capable of replicating indefinitely in cell culture is effected by standard and well-known techniques, for example, by using polyethylene glycol (PEG) or other fusing agents (See Milstein and Kohler, Eur. J. Immunol. 6:511 (1976), which is hereby incorporated by reference in its entirety). This immortal cell line, which is preferably murine, but may also be derived from cells of other mammalian species, including but not limited to rats and humans, is selected to be deficient in enzymes necessary for the utilization of certain nutrients, to be capable of rapid growth and to have good fusion capability. Many such cell lines are known to those skilled in the art, and others are regularly described.

Procedures for raising polyclonal antibodies are also well known. Typically, such antibodies can be raised by administering one of the proteins or polypeptides of the present invention subcutaneously to New Zealand white rabbits which have first been bled to obtain pre-immune serum. The antigens can be injected at a total volume of 100 [mu] 1 per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthenized with pentobarbitol 150 mg/Kg IV. This and other procedures for raising polyclonal antibodies are disclosed in E. Harlow, et. al., editors, Antibodies: A Laboratory Manual (1988), which is hereby incorporated by reference in its entirety.

In addition to utilizing whole antibodies, the processes of the present invention encompass use of binding portions of such antibodies. Such antibody fragments can be made by conventional procedures, such as proteolytic fragmentation procedures, as described in J. Goding, Monoclonal Antibodies: Principles and Practice, pp. 98-118 (N.Y. Academic press 1983), which is hereby incorporated by reference in its entirety.

The vaccines and passive immunization agents of this invention can be administered orally, parenterally, for example, subcutaneously, intravenously, intramuscularly, intraperitoneally, by intranasal instillation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The solid unit dosage forms can be of the conventional type. The solid form can be a capsule, such as an ordinary gelatin type containing the proteins or peptides of the present invention or the antibodies or binding portions thereof of the present invention and a carrier, for example, lubricants and inert fillers such as, lactose, sucrose, or cornstarch. In another embodiment, these compounds are tableted with conventional tablet bases such as lactose, sucrose, or cornstarch in combination with binders like acacia, cornstarch, or gelatin, disintegrating agents such as, cornstarch, potato starch, or alginic acid, and a lubricant like stearic acid or magnesium stearate.

The isolated nucleic acid molecules, proteins, or polypeptides of the present invention or the antibodies or binding portions raise against the proteins or polynucleotides of this invention may also be administered in injectable dosages by solution or suspension of these materials in a physiologically acceptable diluent with a pharmaceutical carrier. Such carriers include sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions.

The present invention also relates to a method of vaccinating mammals against onset of disease caused by infection of Helicobacter bizzozeronii. This method involves administering to mammals an effective amount of at least one of the isolated nucleic acid molecules of the present invention. Suitable techniques for such gene therapy techniques are well known and are described in U.S. Pat. Nos. 5,328,470 and 6,339,068, the entire disclosures of which are hereby incorporated by reference.

For use as aerosols, the proteins or polypeptides of the present invention or the antibodies or binding portions thereof of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

In yet another aspect of the present invention, the proteins or polypeptides of the present invention can be used as antigens in diagnostic assays for the detection of Helicobacter bizzozeronii in body fluids. Alternatively, the detection of that bacterium can be achieved with a diagnostic assay employing antibodies or binding portions thereof raised by such antigens. Such techniques permit detection of Helicobacter bizzozeronii in a sample of the following tissue or body fluids: blood, spinal fluid, sputum, pleural fluids, urine, bronchial alveolor lavage, lymph nodes, bone marrow, or other biopsied materials.

In one embodiment, the assay system has a sandwich or competitive format. Examples of suitable assays include an enzyme-linked immunosorbent assay, a radioimmunoassay, a gel diffusion precipitan reaction assay, an immunodiffusion assay, an agglutination assay, a fluorescent immunoassay, a protein A immunoassay, or an immunoelectrophoresis assay.

In an alternative diagnostic embodiment of the present invention, the nucleotide sequences of the isolated DNA molecules of the present invention may be used as a probe in nucleic acid hybridization assays for the detection of Helicobacter bizzozeronii in various body fluids. The nucleotide sequences of the present invention may be used in any nucleic acid hybridization assay system known in the art, including, but not limited to, Southern blots (Southern, J. Mol. Biol. 98: 503-517 (1975) (which discloses hybridization in 2×SSC (i.e., 0.15M NaCl, 0.015 sodium citrate), 40% formamide at 40 degrees Celsius; Northern blots (Thomas et al., Proc. Nat'l Acad. Sci. USA 77:5201-05 (1980)); Colony blots (Grunstein et al. Proc. Nat'l Acad. Sci. USA 72:3961-65 (1975), which are hereby incorporated by reference in their entirety). Alternatively, the isolated DNA molecules of the present invention can be used in a gene amplification detection procedure (e.g., a polymerase chain reaction). See H. A. Erlich et. al., “Recent Advances in the Polymerase Chain Reaction,” Science 252:1643-51 (1991), which is hereby incorporated by reference in its entirety.

### EXAMPLES

The Examples set forth below are for illustrative purposes only and are not intended to limit, in any way, the scope of the present invention.

### Example 1

### Cloning and Characterization of a Helicobacter bizzozeronii Urease Gene Cluster

The urease gene cluster from Helicobacter bizzozeronii was cloned and sequenced. A genomic library was constructed in a λ-ZAPII vector using TSP5091-digested H. bizzozeronii chromosomal DNA. Four overlapping recombinant bacteriophages carrying the H. bizzozeronii urease genes were identified by using a fragment of H. bizzozeronii ureB as a probe. Sequence analysis of two clones (pHB1 and pHB3) revealed seven open reading frames encoding proteins with predicted masses of 26.5, 60.3, 21.7, 19.5, 28.6, 21.7, and 29.6 kDa representing the structural genes, Urease A and B, and its accessory genes, urease I, E, F, G and H, respectively. In addition, three open reading frames upstream of the ureA gene encoding a putative tRNA transferase, a putative Glucose inhibited division protein B (GidB) and a protein with unknown function were also identified. A clone (pHB5) containing a complete urease gene cluster was constructed. The homologue analysis revealed that UreA polypeptide exhibited 64-90% identity to that of H. heilmannii, H. felis, H. pylori, H. mustelae, and H. hepaticus. UreB polypeptides exhibited 76.8-96% identity to that of H. heilmannii, H. felis, H. pylori, H. mustelae, and H. hepaticus. The UreI, E, F, G and H also showed 44-86% identity to that of H. pylori. Among these accessory genes, UreE had a lowest percentage identity to that of H. pylori.

### Example 2

### Bacterial Strains and Culture Conditions

H. bizzozeronii and H. felis strains were obtained from American Type Cell Culture (ATCC). H. bizzozeronii (ATCC 700030) was cultured on 5% sheep blood agar plates in a microaerophilic gas generating system (Mitsubishi Gas Chemical Company, Inc. Japan) for eight to ten days. H. felis (ATCC 49179) was cultured under the same conditions for six days. E. coli XL1-blue MRF′ XL1, SOLR, Topo10, CQ21 (lacI^{q}), and BL21 (DE3) plysS (Stratagene, Calif.) were grown in Luria-Bertani broth (LB) and on LB agar with appropriate antibiotics (ampicillin 50 μg/ml, kanamycin 50 μg/ml, and chloramphenicol 34 μg/ml).

### Example 3

### Construction of the H. bizzozeronii Library

Chromosomal DNA was purified as previously described in Chang et al., “Identification and Characterization of the Pasteurella haemolytica Leukotoxin,” Infect. Immun. 55:2348-2354 (1987), which is hereby incorporated by reference in its entirety. Chromosomal DNA from H. bizzozeronii was partially digested by a restriction enzyme, Tsp5091, the 3-9 kb DNA fragments were mixed with an EcoRI cut λ-ZAPII vector (Stratagene, La Jolla, Calif.) and treated with T4 DNA ligase for 16 hours at 4° C. The ligated DNA mixture was packaged into λ particles with a commercially available in vitro packaging kit (Gigapack plus, Stratagene). The phage titers were determined and amplified on E. coli MRF′ XL1 blue.

### Example 4

### Phage Library Screening for H. bizzozeronii Urease Gene Cluster

The bacteriophage library was screened by hybridization with a probe containing the ureB gene from H. bizzozeronii. A 500 bp DNA fragment of ureB was amplified and purified by PCR with conserved primers (sense: 5′-ATTACGGGCGATAAAGT-3′ (SEQ ID NO: 16); anti-sense: 5′-CGACTTGGACATCGTAT-3′ (SEQ ID NO: 17)) from H. pylori and H. felis. The DNA fragment was labeled with a non-radioactive labeling kit (ECL™ direct nucleic acid labeling and detection systems, Amersham, Little Chalfont, Buckinghamshire, England) (Chang et al., “Dissemination of Borrelia burgdorferi After Experimental Infection in Dogs,” J. Spirochetal Tick-borne Dis. 3:80-86 (1996), which is hereby incorporated by reference in its entirety). DNA hybridization and detection were performed as described by the manufacturer (ECL™ direct nucleic acid labeling and detection systems, Amersham). Plaques which gave positive signals were picked, rescreened on E. coli MRF′ XL1 blue. Phages isolated from the positive plaques were excised as Bluescript plasmids according to the manufacturer's directions (Stratagene).

### Example 5

### DNA Manipulation

All standard DNA manipulations and analyses, except where mentioned, were performed according to the procedures described in Sambrook et al., Molecular Cloning: A Laboratory Manual Cold Spring Harbor, N.Y.: Cold Spring Harbor Laboratory Press (1989), which is hereby incorporated by reference in its entirety. To construct a complete urease gene cluster, two PCR primers (sense: CCATCAGGGAAGTTAGCTTCAA (SEQ ID NO: 18); anti-sense: CCACTAGTATTTGGTAGAAGGAGTTT (SEQ ID NO: 19)) were designed to amplify a 370 bp fragment using pHB1 as a template and ligated into a PCR Blunt II-Topo vector (Strategene, Calif.). The SpeI-NdeI fragment was digested, purified, and ligated to pHB4 digested with the same restriction enzymes to create pHB5 and transferred into E. coli Topo 10 cells (Strategene, Calif.). The complete urease gene cluster was confirmed by DNA sequencing.

To overexpress the UreB of H. bizzozeronii, the ureB gene was amplified using pHB1 as a template for PCR. PCR was performed using primers (sense 5′-GAATTCATGAAAAATCTCTCGAAA (SEQ ID NO: 20); anti-sense: 5′-AAGCTTCTAGAACAAGTTGTAGAGTTGT (SEQ ID NO: 21)) and PWO polymerase (Boehringer Mannheim, Indianapolis, Ind.) as previously described in Simpson et al., “Helicobacter felis Infection is Associated with Lymphoid Follicular Hyperplasia and Mild Gastritis but Normal Gastric Secretory Function in Cats,” Infect. Immun. 68:779-790 (2000), which is hereby incorporated by reference in its entirety. The PCR products were digested with restriction enzymes, EcoRI and HindIII, and ligated into pKK223-3 (Amersham Pharmacia Biotech, Piscataway, N.J.) cut with the same enzymes to create pHBureB. pHBureB containing ureB gene was transferred into E. coli CQ21.

### Example 6

### Assay for Urease Activity

Qualitative and quantitative urease activity was determined by an enzyme coupled assay with glutamate dehydrogenase and NADH (Kaltwasser et al., “NADH-Dependent Coupled Enzyme Assay for Urease and Other Ammonia-Producing Systems,” Anal. Biochem. 16:132-138 (1966), which is hereby incorporated by reference in its entirety) as previously described for H. bizzozeronii and recombinant urease gene clones (Simpson et al., “Helicobacter felis Infection is Associated with Lymphoid Follicular Hyperplasia and Mild Gastritis but Normal Gastric Secretory Function in Cats,” Infect. Immun. 68:779-790 (2000), which is hereby incorporated by reference in its entirety). Protein concentration was measured with a protein assay kit (Bio-Rad, Calif.) using bovine serum albumin as the standard. H. bizzozeronii were cultured for 10 days, harvested and washed twice in PBS. The recombinant urease clones were cultured in LB broth to OD₆₀₀ nm=0.5, induced by 1 mM IPTG for 2 hours, harvested and washed with PBS. Pellets from both H. bizzozeronii and the recombinant urease clones were re-suspended in PEB buffer {0.1 M sodium phosphate buffer (pH 7.4) containing 0.01 M EDTA} and sonicated with four 30-second bursts using a Branson Sonifier model 450 (Branson Ultrasonic Corporation, Danbury, Conn.) set at 30 W. Cell debris was removed by centrifugation (3,000×g, 10 min). Freshly prepared samples (10 to 20 μl) were added to a reaction mixture containing 3 ml of 31 mM Tris-HCl (pH 8), 810 μM oxoglutarate, 240 μM NADH and 10 mM ureA. Then, 96 U of glutamate dehydrogenease (Boehringer Mannheim, Indianapolis, Ind.) was added to start the reaction. The mixture was incubated at 37° C. for 10 min and the reduction of NADH was measured at 340 nm in a dual beam spectrophotometer (Coulter, Inc., Fullerton, Calif.). Urease activity was expressed in units of μM ammonia min⁻¹mg⁻¹ bacterial protein.

### Example 7

### Sera

Sera were obtained from dogs and cats that had been infected with Helicobacter spp. Sera from uninfected SPF dogs and cats were used as negative controls. The source and generation of these sera have been previously described (Strauss-Ayali et al., “Serological Discrimination of Dogs Infected with Gastric Helicobacter spp. and Uninfected Dogs,” J. Clin. Microbiol. 37:1280-1287 (1999), which is hereby incorporated by reference in its entirety).

### Example 8

### Purification of UreB Protein and Antiserum Production in Rabbit

E. coli CQ21 harboring pHBUreB was grown in LB medium to OD_{600 nm}˜0.5. IPTG was added to a final concentration of 1 mM and the culture was grown for 2 h. The cells were harvested by centrifugation and resuspended in PBS containing 0.5% TritonX-100 and 10 mM EDTA. Cells were ruptured by French press at 8,000 psi. The total lysate was then centrifuged at 3,000×g for 5 minutes to separate cell debris (Chang et al., “Cloning, Sequencing and Expression of a Pasteurella haemolytica A1 Gene Encoding a PurK-Like Protein,” DNA Seq. 3:357-367 (1993), which is hereby incorporated by reference in its entirety) and then centrifuged at 12,000×g for 5 min at 4° C. The pellets were washed with 9 volumes (v/v) 50 mM Tris-HCl, pH 8.0 and 50 mM NaCl containing 0.5% TritonX-100 and 10 mM EDTA, incubated at room temperature for 5 min and then centrifuged at 12,000×g for 5 min at 4° C. The washing step was repeated once. The protein samples were separated by SDS-polyacrylamide gel electrophoresis (SDS-PAGE). Proteins were visualized after staining with Coomassie blue, the rUreB band was excised, mixed with an equal volume of PBS and homogenized for rabbit injection. Polyclonal antiserum to rUreB was raised in New Zealand White rabbits by three subcutaneous injections at three week intervals with approximately 100 μg in Freud's incomplete adjuvant as previously described (Chang et al., “Recombinant OspA Protects Dogs Against Infection and Disease Caused by Borrelia burgdorferi,” Infect. Immun. 63:3543-3549 (1995), which is hereby incorporated by reference in its entirety).

### Example 9

### SDS-PAGE and Western Blotting

The procedures for the SDS-PAGE and Western blot analyses were as previously described (Chang et al., “Cloning, Sequencing and Expression of a Pasteurella haemolytica A1 Gene Encoding a PurK-Like Protein,” DNA Seq. 3:357-367 (1993); and Chang et al., “Molecular Analysis of the Actinobacillus pleuropneumoniae RTX Toxin-III Gene Cluster,” DNA Cell Biol. 12:351-362 (1993), which are hereby incorporated by reference in their entirety). For identification of UreB, the rabbit anti-rUreB anti-serum served as the primary antibody (1:8,000). Goat alkaline phosphatase-conjugated anti-rabbit IgG (KPL, Gaithersburg, Md.) was used as the secondary antibody (1:5,000). To determine whether the naturally infected dog sera contain anti-UreB antibodies, rUreB protein was used as an antigen and subjected to SDS-PAGE and Western blot analysis. Test sera (1:500) from naturally infected animals were used as a first antibody, followed by goat anti-dog or anti-cat IgG conjugated to alkaline phosphate (1:5,000) (KPL, Gaithersburg, Md.) as the secondary reagent.

### Example 10

### 5′ Rapid Amplification of cDNA Ends (5′-RACE)

To determine the transcription start site, 5′-RACE was followed as previously described with modification (Frohman, M. A., “Rapid Amplification of Complementary DNA Ends for Generation of Full-Length Complementary DNAs: Thermal RACE,” Methods Enzymol. 218:340-356 (1993), which is hereby incorporated by reference in its entirety). Total RNA from H. bizzozeronii was isolated using TRIzol (Gibco-BRL) from 8 day culture plates. The RNA obtained was treated with amplification-grade DNaseI (Gibco-BRL) and reverse transcription PCR (RT-PCR) was performed using SuperScript™ II Reverse Transcriptase (Gibco-BRL) according to the manufacturer's recommendations. The first strand cDNA (antisense) was synthesized by using Hb-specific primers (uAGSP1: TTCCAGAAGCAATGTCTAGG (SEQ ID NO: 22), uIGSPI: CCATCATCGAAGGTGTGATC (SEQ ID NO: 23)). The RNA template was then degraded with RNase H (Gibco-BRL) and the single-strand cDNA was purified with the PCR Purification Kit (Gibco-BRL). An oligo-dC anchor sequence was added to the 3′ end of the cDNA using TdT (terminal deoxynucleotidyl transferase) and dCTP (deoxycytidine triphosphate). PCR amplification was accomplished with two primers: A deoxyinosine-containing anchor primer Qc (5′-CCAGTGAGCAGAGTGACGAGGACTCGAGCTCAAG CTTGGGIIGGGIIGGGIIG-3′ (SEQ ID NO: 24)) that annealed to the poly (G) tail of the cDNA and a nested hb-specific primer (uAGSP2: AAAGAAGTGGA AGTGGGAGC (SEQ ID NO: 25), uIGSP2: GCACATTCAATCCAACCAGT (SEQ ID NO: 26)). Following that, a second-round nested PCR was performed with the primer Qo (CCAGTGAGCAGAGTG ACG (SEQ ID NO: 27)), Qi (GAGGACTCGAGCTCAAGC (SEQ ID NO: 28)) which was complementary to the 5′ end of Qc, and the other hb-specific primers (uAGSP3:CATTCAAAGTG ATGTCTTCGTT (SEQ ID NO: 29), uIGSP3: GGCTTGATCATCAAGTGCA TCTGAGT (SEQ ID NO: 30)). The resulting PCR product was cloned into pCR2.1 vector (Invitrogen, Calif.) and subjected to DNA sequencing. Three different clones were sequenced to determine the transcription start site. The experiment was repeated three times.

### Example 11

### Nucleotide Sequence Accession Number

The DNA sequence of ureABIEFGH has been submitted to Genbank and assigned accession number AF 330621.

### Example 12

### Cloning and Sequencing of H. bizzozeronii Urease Gene Cluster

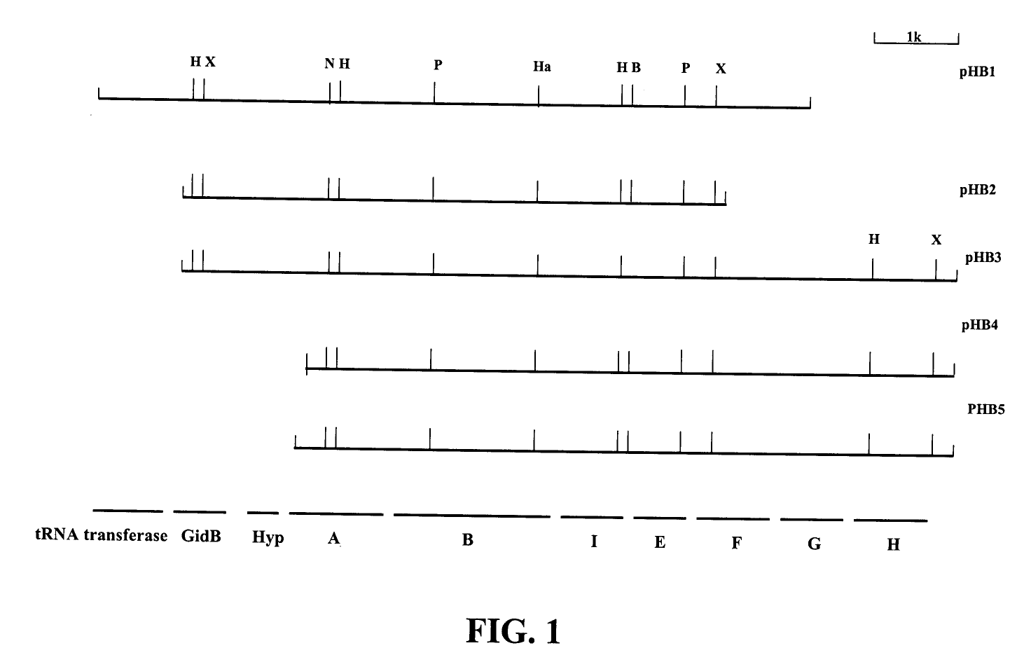

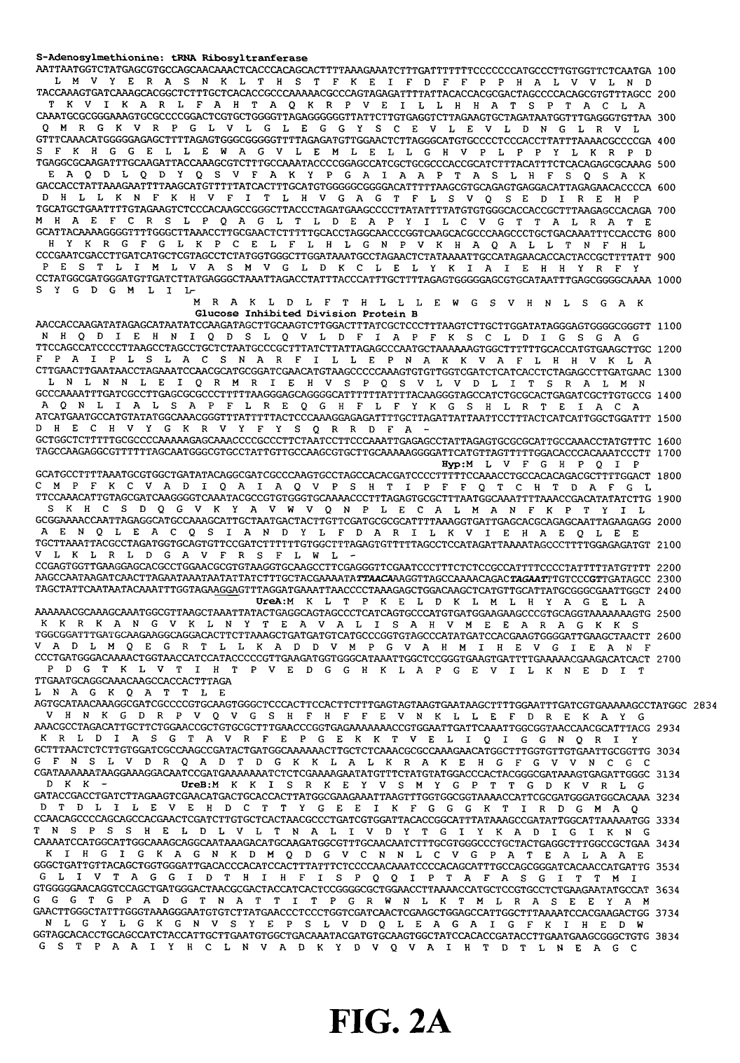

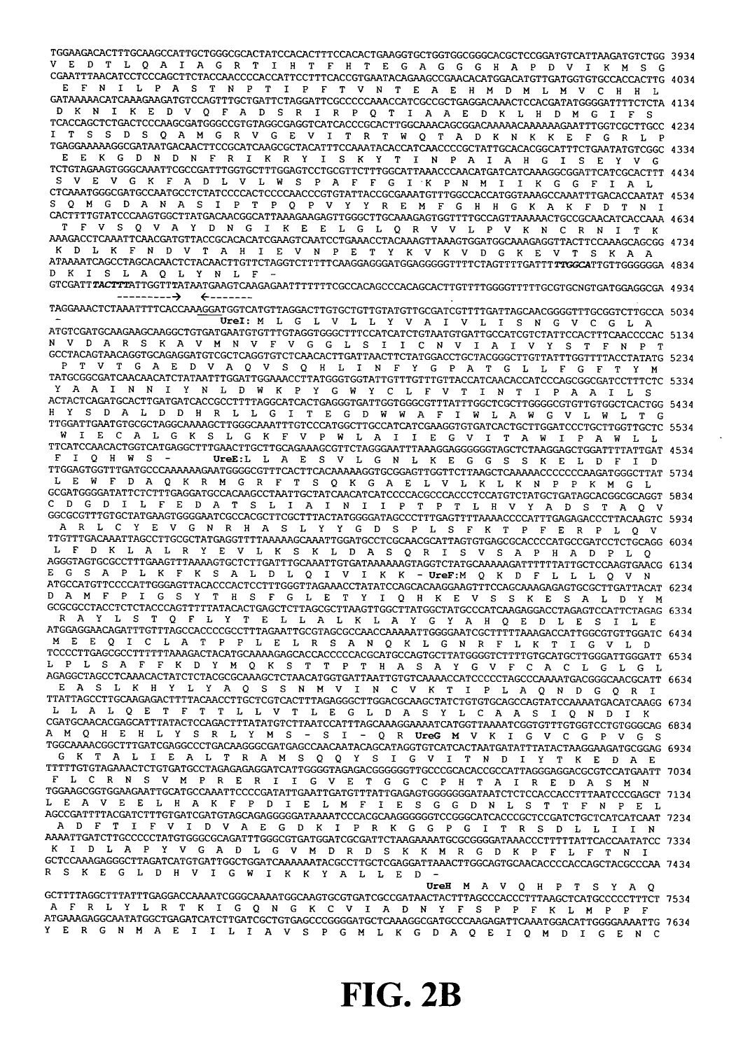

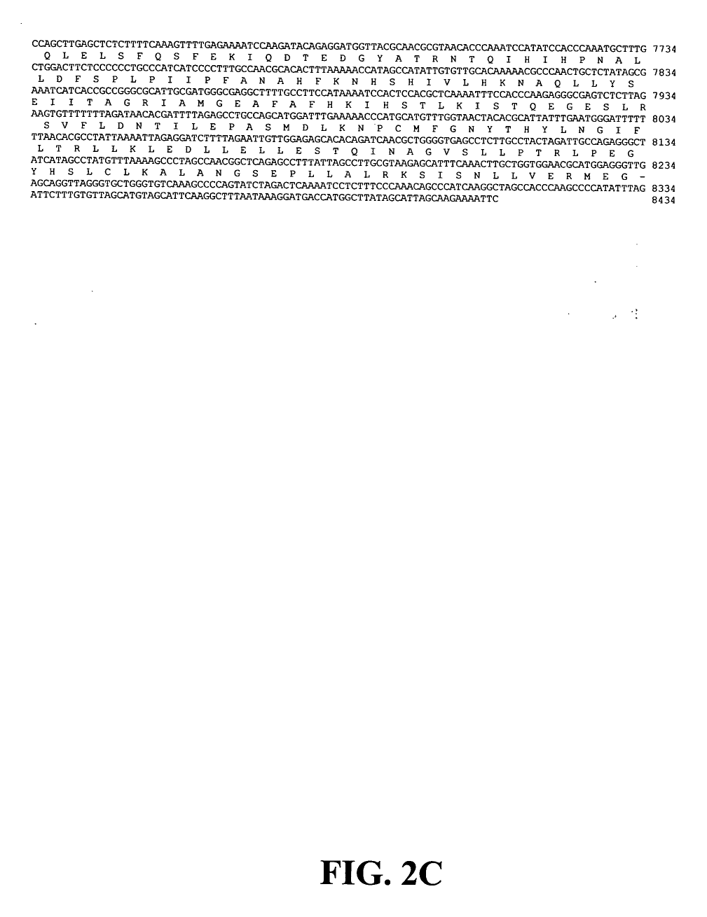

A Helicobacter bizzozeronii genomic library constructed in the phage vector λ-ZapII with a 500 bp DNA probe of ureB amplified from H. bizzozeronii genomic DNA by PCR. Ten positive plaques were identified from 10,000 plaques. Four clones were isolated which overlapped each other (FIG. 1). DNA from pHB1 and pHB3 were subjected to DNA sequence analysis (FIGS. 2A-2C). As in the case of the H. pylori urease loci, there are 7 open reading frames (ORFs), presumably encoding the ureA, B, I, E, F, G, and H genes (FIGS. 2A-2C) There are three ORFs upstream of the ureA gene, encoding the tRNA transferase (SEQ ID NO:31), GidB (SEQ ID NO:32) and an ORF with unknown function denoted as Hyp (SEQ ID NO:33). The ureA, B, I, E, F, G, and H genes are closely related to those of H. pylori. Table I summarizes the similarities among H. bizzozeronii, H. heilmanni, H. pylori, H. felis, H. mustelae, and H. hepaticus ureAB genes and among H. bizzozeronii, H. pylori and H. hepaticus ureIEFGH genes.

TABLE 1
Homology of the Urease Gene Cluster of
Helicobacter bizzozeronii With Other Helicobacter Species

| COMPARISON | PERCENT (%) IDENTITY OF: | | | | | | |
|---|---|---|---|---|---|---|---|
| SPECIES | UreA | UreB | UreI | UreE | UreF | UreG | UreH |
| H. heilmannii | 90.2 | 96.0 | | | | | |
| H. felis | 82.9 | 93.7 | | | | | |
| H. pylori | 76.1 | 88.1 | 77.4 | 44.1 | 65.8 | 86.4 | 49.4 |
| H. hepaticus | 64.0 | 76.8 | 58.2 | 31.0 | 52.7 | 82.9 | 44.8 |
| H. mustelae | 70.4 | 77.3 | | | | | |

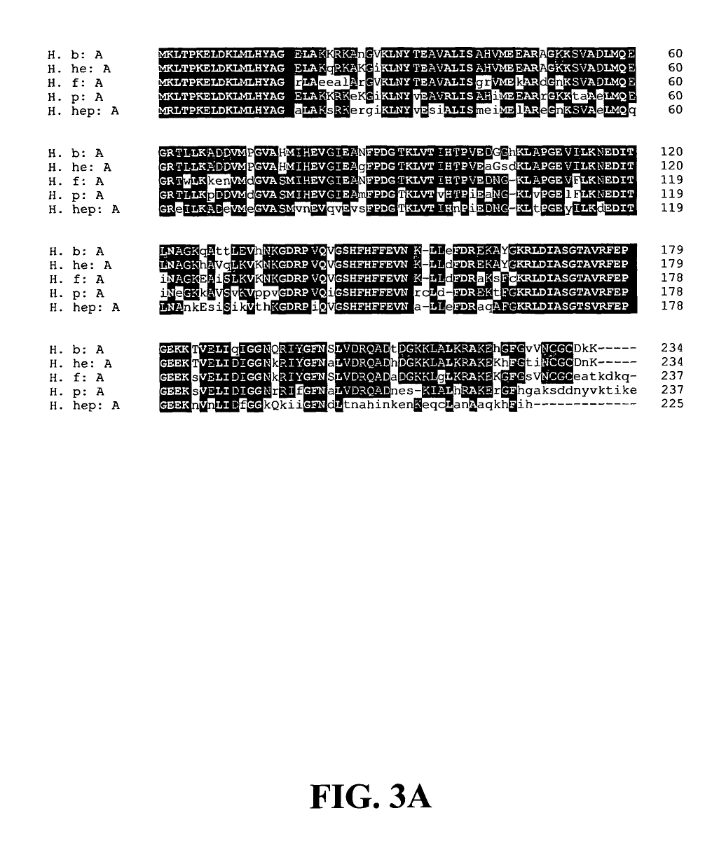

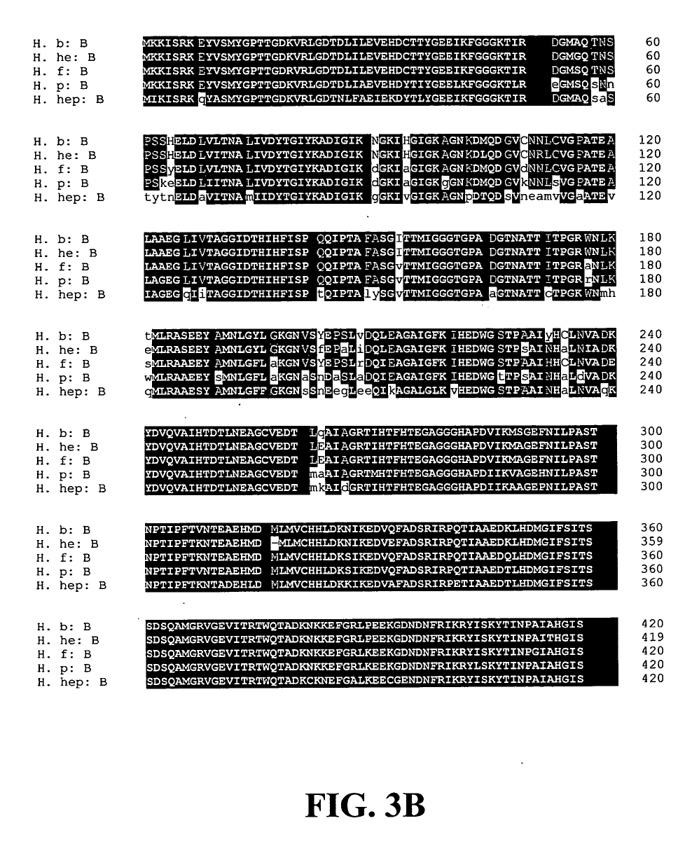

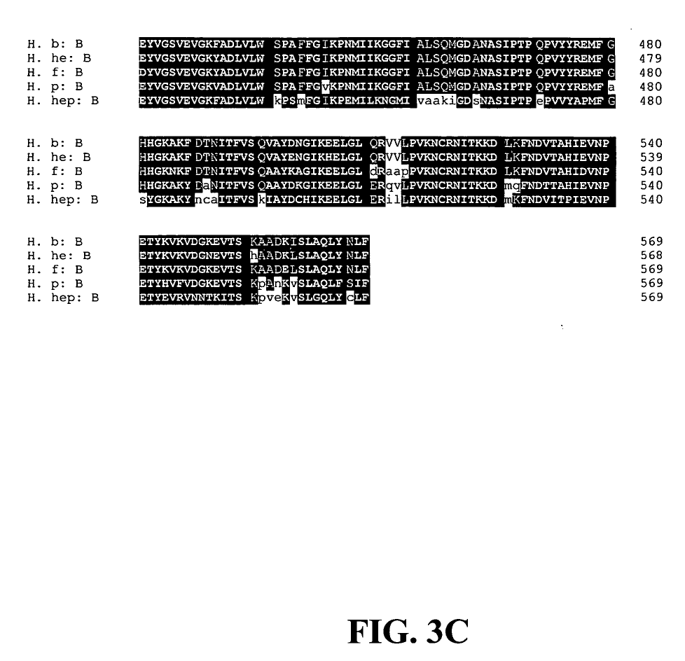

The level of UreA/UreB amino acid identity is 90.2%/96%, 82.9%/93.7%, 76.1%/88.1%, 70.4%/77.3%, and 64%/76.8% to that of H. heilmannii, H. felis, H. pylori, H. mustelae, and H. hepaticus, respectively (Table. 1 and FIGS. 3A-3C). The urease accessory gene products, Ure I (SEQ ID NO:15), UreE (SEQ ID NO:7), UreF (SEQ ID NO:9), UreG (SEQ ID NO:11), and UreH (SEQ ID NO:13), showed 77.4%, 44.1%, 65.8%, 86.4%, and 49.4% identity to that of H. pylori, and 58.2%, 31%, 52.7%, 82.9%, and 44.8% identity to that of H. hepaticus (Table land FIGS. 2A-2C). UreE showed the lowest identity among these genes (44%).

TTG codons initiated ureE and ureH ORFs and ATG codons initiated the rest of the ORFs of the urease gene cluster. The intergenic region between ureB and ureI contains about 188 bp. This structure is similar to that of H. pylori (192 bp) but different from H. hepaticus which contains only 9 bp between the ureB and ureI genes. However, the intergenic regions between ureB and ureI DNA sequences of H. pylori and H. bizzozeronii are heterologous. The ureAB sequence was examined for E. coli promoter-like sequences by the homology score method (Mulligan et al., “Analysis of the Occurrence of Promter-Sites in DNA,” Nucl. Acid Res. 12:789-800 (1984), which is hereby incorporated by reference in its entirety). There was a sequence, TAGAAT, similar to the TATAAT consensus promoter sequence (−10 region) and one sequence, TTAACA, similar to the consensus RNA potential ribosome-binding site (−35 region) proximal to ureA (FIGS. 2A-2C).

### Example 13

### 5′-RACE for Determination of the Transcript Start Site

Based on the sequencing results, the transcriptional start site was located at G/T and T nucleotides downstream from the promoter −10 region for ureA and ureI, respectively (FIGS. 2A-2C).

### Example 14

### Expression of H. bizzozeronii UreB in Recombinant Clones and Western Blot Analysis

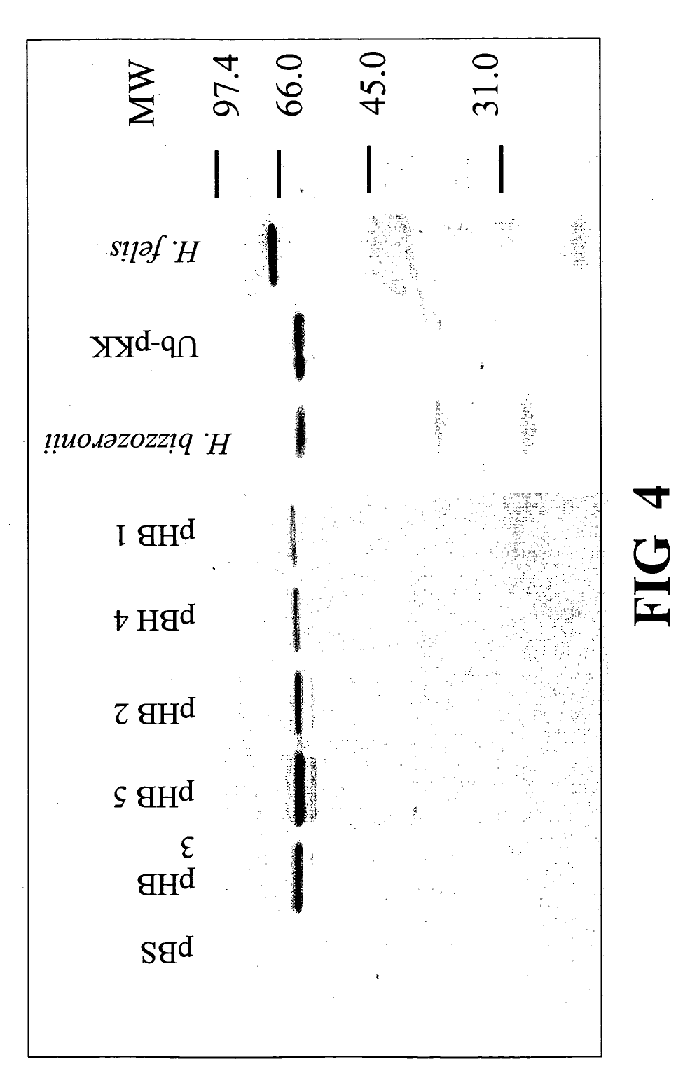

Western blot results indicated that in E. coli clones containing different lengths of the urease gene cluster sequence and a clone harboring only ureB gene, UreB was expressed at a level compatible with that of wild type H. bizzozeronii bacteria and had a tentative molecular mass of 60 kDa. The protein mass is smaller than that of H. felis, 66 kDa (FIG. 4). The recombinant protein from the clone containing pHBUreB was partially purified from E. coli lysates by washing with Triton-100 since it formed insoluble inclusion bodies. UreB was expressed in clones containing pHB1 to pHB5, and the expression level in clone containing pHB5 was higher than that of the other four clones (FIG. 4).

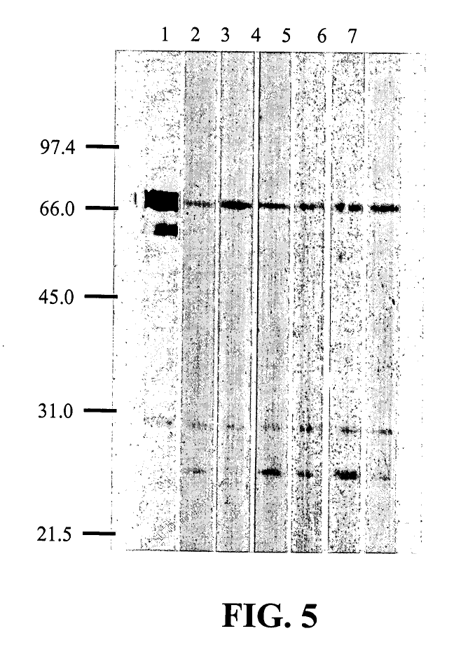

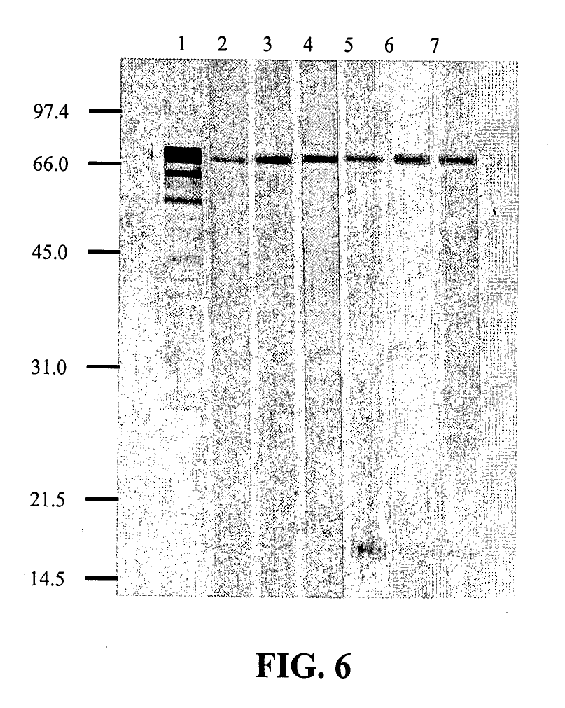

To evaluate whether naturally infected dog sera contained anti-UreB antibodies, five naturally plus three uninfected dog and cat sera each were used for Western blot analysis. The results showed that partially purified rUreB was recognized by the naturally H. bizzozeronii-infected dog and cat antisera, but also recognized by the normal serum or the SPF dog's sera (FIG. 5 and FIG. 6).

### Example 15

### Urease Activity Assays

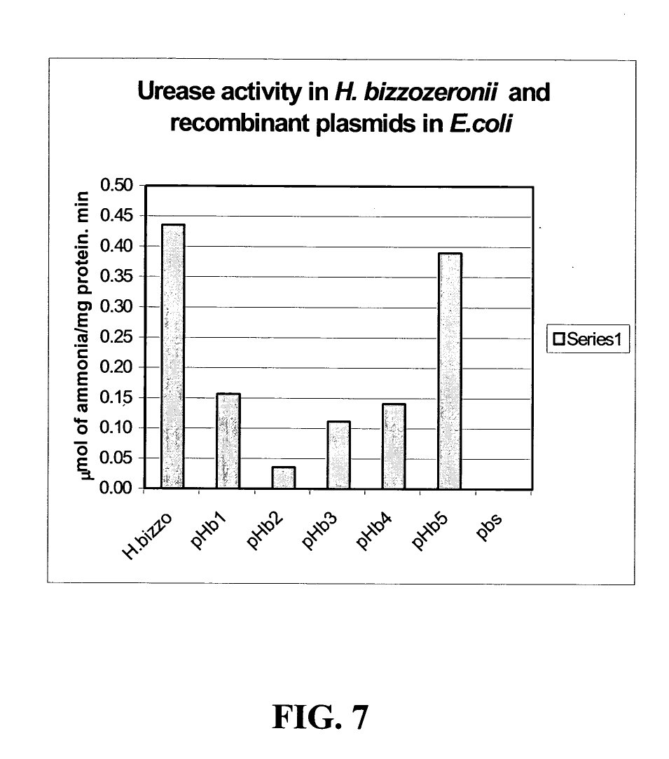

Both qualitative and quantitative urease activities were measured (FIG. 7). The E. coli containing only UreB protein did not show any urease activity. For the other clones with recombinant proteins, the one with whole gene cluster showed the highest activity. Clones (pHB1, pHB2, pHB3, and pHB4) showed a very low urease activity (Student T-test, p<0.05).

H. bizzozeronii, a large gastric spiral bacterium, has been isolated from dogs and cats (Hanninen et al., “Culture and Characteristics of Helicobacter bizzozeronii, a New Canine Gastric Helicobacter sp [published erratum appears in Int. J. Syst. Bacteriol. 46(3):839 (1996)] Int. J. Syst. Bacteriol. 46:160-166 (1996), which is hereby incorporated by reference in its entirety) and this organism may be transmitted from dogs to people (Solnick et al., “Emergence of Diverse Helicobactere Species in the Pathogenesis of Gastric and Enterohepatic Diseases,” Clin. Microbiol. Rev. 14:59-97 (2001), which is hereby incorporated by reference in its entirety). Since urease is pivotal in Helicobacter colonization and pathogenesis (Eaton et al., “In Vivo Complementation of ureB Restores the Ability of Helicobacter pylori to Colonize,” Infect. Immun. 70:771-778 (2002); and Mobley et al., “Molecular Biology of Microbial Ureases,” Microbiol. Rev. 59:451-480 (1995), which are hereby incorporated by reference in their entirety), it is important to know whether H. bizzozeronii has a similar urease gene structure. Furthermore, cloning and characterization of the urease gene and its protein should aid the development a recombinant vaccine that might provide cross protection against Helicobacter spp. infection.

The H. bizzozeronii urease gene cluster, similar to that of H. pylori, contains urease structural genes (ureAB) and accessory genes (ureIEFGH) (Mobley et al., “Molecular Biology of Microbial Ureases,” Microbiol. Rev. 59:451-480 (1995), which is hereby incorporated by reference in its entirety). However, there are three ORFs upstream of the ureA gene encoding tRNA transferase, GidB and a protein with unknown function (this study). In contrast, the ORF immediately upstream the ureA gene in H. pylori is a lipoprotein signal peptidase A (Akada et al., “Identification of the Urease Operon in Helicobacter pylori and Its Control by mRNA Decay in Response to pH,” Mol. Microbiol. 36:1071-1084 (2000), which is hereby incorporated by reference in its entirety). Alignment of the UreA and UreB sequences from H. bizzozeronii with that of H. heilmannii, H. pylori and H. felis reveals 76-96% identity. This indicates that they may come from the same ancestor. Among these three species, H. bizzozeronii showed 96% identity to that of H. heilmanii. Recently, it has been reported that a similar bacterium named H. heilmanii was identified in gastric biopsies from people with gastritis (Solnick et al., “Emergence of Diverse Helicobactere Species in the Pathogenesis of Gastric and Enterohepatic Diseases,” Clin. Microbiol. Rev. 14:59-97 (2001), which is hereby incorporated by reference in its entirety). Since there is only one H. heilmanii strain isolated from people so far, it is difficult to prove whether H. bizzozeronii and H. heilmanii are similar organisms that can be transmitted and cause gastritis in both humans and animals (Solnick et al., “Emergence of Diverse Helicobactere Species in the Pathogenesis of Gastric and Enterohepatic Diseases,” Clin. Microbiol. Rev. 14:59-97 (2001), which is hereby incorporated by reference in its entirety).

The H. pylori urease gene cluster consists of two operons, ureAB and ureIEFGH (Akada et al., “Identification of the Urease Operon in Helicobacter pylori and Its Control by mRNA Decay in Response to pH,” Mol. Microbiol. 36:1071-1084 (2000); and Alm et al., “Genomic-Sequence Comparison of Two Unrelated Isolates of the Human Gastric Pathogen Helicobacter pylori, [published erratum appears in Nature 397(6721):719 (1999)] Nature 397:176-180 (1999), which are hereby incorporated by reference in their entirety). H. pylori urease is a 550 kDa enzyme, consisting of two distinct subunits with molecular masses of 29.5 kDa (UreA) and 66 kDa (UreB) at the ratio of 1:1 and suggesting a stoichiometry of (29.5-66 kDa)₆ for the native enzyme (Dunn et al. “Purification and Characterization of Urease from Helicobacter pylori,” J. Biol. Chem. 265:9464-9469 (1990); Evans et al., “Characterization of the Helicobacter pylori Urease and Purification of Its Subunits,” Microb. Pathog. 10:15-26 (1991); and Hu et al., “Purification of Recombinant Helicobacter pylori Urease Apoenzyme Encoded by ureA and ureB,” Infect. Immun. 60:2657-2666 (1992), which are hereby incorporated by reference in their entirety). However, the molecular mass of H. bizzozeronii is approximately 60 kDa (this study). Reportedly, four genes (ureE, ureF, ureG and ureH) located downstream of structural genes ureA and ureB are required for recombinant H. pylori urease activity in E. coli (Cussac et al., “Expression of Helicobacter pylori Urease Genes in Escherichia coli Grown Under Nitrogen-Limiting Conditions,” J. Bacteriol. 174:2466-2473 (1992), which is hereby incorporated by reference in its entirety). Also, the H. pylori urease activity expressed in E. coli is usually weak (<1 μmol of ureA per min per mg of protein after 3 days of growth) (Cussac et al., “Expression of Helicobacter pylori Urease Genes in Escherichia coli Grown Under Nitrogen-Limiting Conditions,” J. Bacteriol. 174:2466-2473 (1992), which is hereby incorporated by reference in its entirety). It was also found that H. bizzozeronii urease activity is very low when expressed in E. coli. To achieve catalytically active urease when expressed in E. coli, it was suggested that the E. coli containing the urease structural subunit/accessory genes (ureAB/EFGH) be cultured with additional NiCl₂ in minimum medium containing no Ni²⁺-chelating amino acids to make them available for intracellular transport (Hu et al., “Expression of Catalytically Active Recombinant Helicobacter pylori Urease at Wild-Type Levels in Escherichia coli,” Infect. Immun. 61:2563-2569 (1993), which is hereby incorporated by reference in its entirety). It has been also reported that co-expression of the high-affinity nickel transport protein, NixA, in E. coli carrying the H. pylori urease gene, might enhance urease activity by facilitating nickel transport into the cell (McGee et al., “Isolation of Helicobacter pylori Genes that Modulate Urease Activity,” J. Bacteriol. 181:2477-2484 (1999); and Mobley et al., “Helicobacter pylori Nickel-Transport Gene nixA: Synthesis of Catalytically Active Urease in Escherichia coli Independent of Growth Conditions,” Mol. Microbiol. 16:97-109 (1995), which are hereby incorporated by reference in their entirety). Urease gene expression is regulated by nickel at the transcriptional level (van Vliet et al., “Nickel-Responsive Induction of Urease Expression in Helicobacter pylori is Mediated at the Transcriptional Level,” Infect. Immun. 69:4891-4897 (2001), which is hereby incorporated by reference in its entirety). Similarly, since culturing was only in LB without the addition of nickel and/or co-transforming with the nixA gene in E. coli, the data showed a very low urease activity.

UreI from H. bizzozeronii was 77.4% homologous to that of H. pylori. UreI is essential for activation of cytoplasmic urease at low pH (pH<4.0), but is not required for biogenesis of active urease (Rektorschek et al., “Acid Resistance of Helicobacter pylori Depends on the UreI Membrane Protein and an Inner Membrane Proton Barrier,” Mol. Microbiol. 36:141-152 (2000); Scott et al., “Expression of the Helicobacter pylori ureI Gene is Required for Acidic pH Activation of Cytoplasmic Urease,” Infect. Immun. 68:470-477 (2000); and Skouloubris et al., “The Helicobacter pylori UreI Protein is Not Involved in Urease Activity but is Essential for Bacterial Survival in vivo,” Infect. Immun. 66:4517-4521 (1998), which are hereby incorporated by reference in their entirety). UreI has properties similar to those of a highly selective H⁺-gated urea channel (Weeks et al., “A H+-Gated Urea Channel: The Link Between Helicobacter pylori Urease and Gastric Colonization,” Science 287:482-485 (2000), which is hereby incorporated by reference in its entirety). Since ureA is an amide and UreI is homologous to other putative amide transporters (Chebrou et al., “Amide Metabolism: A Putative ABC Transporter in Rhodococcus sp. R312,” Gene 182:215-218 (1996); Wilson et al., “Identification of Two New Genes in the Pseudomonas aeruginosa Amidase Operon, Encoding an ATPase (AmiB) and a Putative Integral Membrane Protein (AmiS),” J. Biol. Chem. 270:18818-18824 (1995), which are hereby incorporated by reference in their entirety), UreI may be a member of an amidoporin family of transporters (Rektorschek et al., “Acid Resistance of Helicobacter pylori Depends on the UreI Membrane Protein and an Inner Membrane Proton Barrier,” Mol. Microbiol. 36:141-152 (2000), which is hereby incorporated by reference in its entirety). The DNA sequence of the ureI promoter of H. bizzozeronii was compared to that of H. pylori. Unlike H. pylori, there is an incomplete inverted repeat sequence upstream from the ureI transcription start point (Akada et al., “Identification of the Urease Operon in Helicobacter pylori and Its Control by mRNA Decay in Response to pH,” Mol. Microbiol. 36:1071-1084 (2000), which is hereby incorporated by reference in its entirety). Also, the intergenic nucleotide sequences between ureB and ureI of H. bizzozeronii and H. pylori are 48% homologous.

A gene encoding a hypothetical protein (Hyp) located upstream of the urease gene cluster of H. bizzozeronii was identified. This gene is similar to a H. pylori gene whose function is also unknown, but the Hyp gene in H. pylori is located at upstream of the ureA. A putative periplasmic-like protein (ORF P) was identified upstream of the ureA in H. hepaticus also (Beckwith et al., “Cloning, Expression, and Catalytic Activity of H. hepaticus Urease,” Infect. Immun. 69:5914-5920 (2001), which is hereby incorporated by reference in its entirety). Downstream of ureH is a nickel transport system and a hypothetical protein in H. hepaticus and H. pylori, respectively (Alm et al., “Genomic-Sequence Comparison of Two Unrelated Isolates of the Human Gastric Pathogen Helicobacter pylori, [published erratum appears in Nature 397(6721):719 (1999)] Nature 397:176-180 (1999); and Beckwith et al., “Cloning, Expression, and Catalytic Activity of H. hepaticus Urease,” Infect. Immun. 69:5914-5920 (2001), which are hereby incorporated by reference in their entirety). However, the region downstream of ureH in H. bizzozeronii has not been sequenced.

Repeated 5′-RACE indicated the ureA transcription start site is either a G or T located downstream of −10 promoter (FIGS. 2A-2C). Since the G is located at the corresponding G of the template and also adjacent to the appended poly G tail, it is difficult to say whether the G is derived from poly C addition during 5′-RACE or from the cDNA. However, this G or T is in good agreement with the consensus spacing of 6 to 8 bp between the promoter and transcription start site. The transcription start site of the ureI is T located downstream of −10 promoter (FIGS. 2A-2C).

Western blot analysis used rUreB and the sera obtained from dogs naturally infected with Helicobacter spp. or normal dog's sera recognized rUreB. This is in agreement with previous reports that normal animal sera react to UreB (Strauss-Ayali et al., “Serological Discrimination of Dogs Infected with Gastric Helicobacter spp. and Uninfected Dogs,” J. Clin. Microbiol. 37:1280-1287 (1999), which is hereby incorporated by reference in its entirety). Ureases from different bacteria have some degree of homology (Mobley et al., “Molecular Biology of Microbial Ureases,” Microbiol. Rev. 59:451-480 (1995), which is hereby incorporated by reference in its entirety) and most animals probably harbor normal bacterial flora in their intestine that also secrete urease into their host.

In conclusion, the H. bizzozeronii urease gene cluster has been cloned and sequenced. Since the urease derived from H. bizzozeronii is highly homologous to that of H. pylori and H. felis, it may play a similar role in the pathogenesis of helicobacteriosis in both people and animals. However, it remains to be determined if recombinant urease from H. bizzozeronii could induce cross protection against infection and disease by Helicobacter spp.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. An isolated nucleic acid molecule conferring on Helicobacter bizzozeronii an ability to produce urease, wherein said nucleic acid molecule is a urease gene cluster comprising at least one urease structural gene and at least one urease accessory gene.

2. An isolated nucleic acid molecule according to claim 1, wherein said urease gene cluster comprises the nucleotide sequence corresponding to SEQ ID NO: 1.

3. An isolated nucleic acid molecule from a Helicobacter bizzozeronii urease gene cluster, said nucleic acid molecule being selected from the group consisting of ureA, ureB, ureE, ureF, ureG, ureH, and ureI.

4. An isolated nucleic acid molecule according to claim 3, wherein the isolated nucleic acid molecule is ureA.

5. An isolated nucleic acid molecule according to claim 4, wherein the nucleic acid molecule: encodes a protein or polypeptide having an amino acid sequence of SEQ ID NO: 3; has a nucleotide sequence of SEQ ID NO: 2; or has a nucleotide sequence comprising at least 20 contiguous nucleic acid residues that hybridize to a nucleic acid having a nucleotide sequence of SEQ ID NO: 2 under stringent conditions comprising 50 percent formamide at 42 degrees Celsius.

6. An isolated nucleic acid molecule according to claim 3, wherein the isolated nucleic acid molecule is ureB.

7. An isolated nucleic acid molecule according to claim 6, wherein the nucleic acid molecule: encodes a protein or polypeptide having an amino acid sequence of SEQ ID NO: 5; has a nucleotide sequence of SEQ ID NO: 4; or has a nucleotide sequence comprising at least 20 contiguous nucleic acid residues that hybridize to a nucleic acid having a nucleotide sequence of SEQ ID NO: 4 under stringent conditions comprising 50 percent formamide at 42 degrees Celsius.

8. An isolated nucleic acid molecule according to claim 3, wherein the isolated nucleic acid molecule is ureE.

9. An isolated nucleic acid molecule according to claim 8, wherein the nucleic acid molecule: encodes a protein or polypeptide having an amino acid sequence of SEQ ID NO: 7; has a nucleotide sequence of SEQ ID NO: 6; or has a nucleotide sequence comprising at least 20 contiguous nucleic acid residues that hybridize to a nucleic acid having a nucleotide sequence of SEQ ID NO: 6 under stringent conditions comprising 50 percent formamide at 42 degrees Celsius.

10. An isolated nucleic acid molecule according to claim 3, wherein the isolated nucleic acid molecule is ureF.

11. An isolated nucleic acid molecule according to claim 10, wherein the nucleic acid molecule: encodes a protein or polypeptide having an amino acid sequence of SEQ ID NO: 9; has a nucleotide sequence of SEQ ID NO: 8; or has a nucleotide sequence comprising at least 20 contiguous nucleic acid residues that hybridize to a nucleic acid having a nucleotide sequence of SEQ ID NO: 8 under stringent conditions comprising 50 percent formamide at 42 degrees Celsius.

12. An isolated nucleic acid molecule according to claim 3, wherein the isolated nucleic acid molecule is ureG.

13. An isolated nucleic acid molecule according to claim 12, wherein the nucleic acid molecule: encodes a protein or polypeptide having an amino acid sequence of SEQ ID NO: 11; has a nucleotide sequence of SEQ ID NO: 10; or has a nucleotide sequence comprising at least 20 contiguous nucleic acid residues that hybridize to a nucleic acid having a nucleotide sequence of SEQ ID NO: 10 under stringent conditions comprising 50 percent formamide at 42 degrees Celsius.

14. An isolated nucleic acid molecule according to claim 3, wherein the isolated nucleic acid molecule is ureH.

15. An isolated nucleic acid molecule according to claim 14, wherein the nucleic acid molecule: encodes a protein or polypeptide having an amino acid sequence of SEQ ID NO: 13; has a nucleotide sequence of SEQ ID NO: 12; or has a nucleotide sequence comprising at least 20 contiguous nucleic acid residues that hybridize to a nucleic acid having a nucleotide sequence of SEQ ID NO: 12 under stringent conditions comprising 50 percent formamide at 42 degrees Celsius.

16. An isolated nucleic acid molecule according to claim 3, wherein the isolated nucleic acid molecule is ureI.

17. An isolated nucleic acid molecule according to claim 16, wherein the nucleic acid molecule: encodes a protein or polypeptide having an amino acid sequence of SEQ ID NO: 15; has a nucleotide sequence of SEQ ID NO: 14; or has a nucleotide sequence comprising at least 20 contiguous nucleic acid residues that hybridize to a nucleic acid having a nucleotide sequence of SEQ ID NO: 14 under stringent conditions comprising 50 percent formamide at 42 degrees Celsius.

18. A method of vaccinating mammals against onset of diease caused by infection of Helicobacter bizzozeronii comprising:
administering to mammals an effective amount of at least one isolated nucleic acid molecule according to claim 3.

19. The method according claim 18, wherein said administering is oral, intradermal, intramuscular, intraperitoneal, intravenous, subcutaneous, or intranasal.

20. An isolated protein or polypeptide encoded by a nucleic acid molecule according to claim 3.

21. An isolated protein or polypeptide according to claim 20, wherein said protein or polypeptide is recombinant.

22. An isolated protein or polypeptide according to claim 20, wherein said protein or polypeptide is purified.

23. An isolated protein or polypeptide according to claim 20, wherein said protein or polypeptide is encoded by the ureA gene and has an amino acid sequence of SEQ ID NO: 3 or is encoded by a nucleic acid molecule having a nucleotide sequence of SEQ ID NO: 2.

24. An isolated protein or polypeptide according to claim 20, wherein said protein or polypeptide is encoded by the ureB gene and has an amino acid sequence of SEQ ID NO: 5 or is encoded by a nucleic acid molecule having a nucleotide sequence of SEQ ID NO: 4.

25. An isolated protein or polypeptide according to claim 20, wherein said protein or polypeptide is encoded by the ureE gene and has an amino acid sequence of SEQ ID NO: 7 or is encoded by a nucleic acid molecule having a nucleotide sequence of SEQ ID NO: 6.

26. An isolated protein or polypeptide according to claim 20, wherein said protein or polypeptide is encoded by the ureF gene and has an amino acid sequence of SEQ ID NO: 9 or is encoded by a nucleic acid molecule having a nucleotide sequence of SEQ ID NO: 8.

27. An isolated protein or polypeptide according to claim 20, wherein said protein or polypeptide is encoded by the ureG gene and has an amino acid sequence of SEQ ID NO: 11 or is encoded by a nucleic acid molecule having a nucleotide sequence of SEQ ID NO: 10.

28. An isolated protein or polypeptide according to claim 20, wherein said protein or polypeptide is encoded by the ureH gene and has an amino acid sequence of SEQ ID NO: 15 or is encoded by a nucleic acid molecule having a nucleotide sequence of SEQ ID NO: 14.

29. An isolated protein or polypeptide according to claim 20, wherein said protein or polypeptide is encoded by the ureI gene and has an amino acid sequence of SEQ ID NO: 15 or is encoded by a nucleic acid molecule having a nucleotide sequence of SEQ ID NO: 14.

30. A method of vaccinating mammals against infection by Helicobacter bizzozeronii comprising:
administering to mammals an effective amount of at least one isolated protein or polypeptide according to claim 20.

31. The method according claim 30, wherein said administering is oral, intradermal, intramuscular, intraperitoneal, intravenous, subcutaneous, or intranasal.

32. A recombinant DNA expression system comprising an expression vector into which is inserted a heterologous DNA molecule according to claim 3.

33. A recombinant DNA expression system according to claim 32, wherein said heterologous DNA molecule is inserted into said vector in proper orientation and correct reading frame.

34. A host cell incorporating a heterologous DNA molecule according to claim 3.

35. A host cell according to claim 34, wherein said nucleic acid molecule is inserted in a recombinant DNA expression system comprising an expression vector.

36. A vaccine for preventing onset of disease in mammals infected by Helicobacter bizzozeronii comprising:
an isolated protein or polypeptide according to claim 20; and
a pharmaceutically-acceptable carrier.

37. A method of vaccinating mammals against onset of disease resulting from infection by Helicobacter bizzozeronii comprising:
administering to mammals an effective amount of the vaccine according to claim 36.

38. The method according claim 37, wherein said administering is oral, intradermal, intramuscular, intraperitoneal, intravenous, subcutaneous, or intranasal.

39. An isolated antibody or binding portion thereof raised against a protein or polypeptide according to claim 20.

40. An isolated antibody or binding portion according to claim 39, wherein said antibody is monoclonal or polyclonal.

41. A method of passively immunizing mammals infected with Helicobacter bizzozeronii comprising:
administering an effective amount of said antibody or binding portion thereof according to claim 39 to mammals infected with Helicobacter bizzozeronii.

42. A method according to claim 41, wherein said administering is oral, intradermal, intramuscular, intraperitoneal, intravenous, subcutaneous, or intranasal.

43. A composition for passively immunizing mammals infected with Helicobacter bizzozeroni comprising:
an isolated antibody or binding portion thereof according to claim 39; and
a pharmaceutically-acceptable carrier.

44. A composition according to claim 43, wherein said antibody is monoclonal or polyclonal.

45. A method of passively immunizing mammals infected with Helicobacter bizzozeroni comprising:
administering an effective amount of said composition according to claim 43 to mammals infected with Helicobacter bizzozeronii.

46. A method according to claim 45, wherein said administering is oral, intradermal, intramuscular, intraperitoneal, intravenous, subcutaneous, or intranasal.

47. A method for detection of Helicobacter bizzozeronii in a sample of tissue or body fluids comprising:
providing a protein or polypeptide according to claim 20 as an antigen;
contacting the sample with the antigen; and
detecting any reaction which indicates that Helicobacter bizzozeronii is present in the sample using an assay system.

48. The method according to claim 47, wherein the assay system is selected from the group consisting of an enzyme-linked immunosorbent assay, a radioimmunoassay, a gel diffusion precipitin reaction assay, an immunodiffusion assay, an agglutination assay, a fluorescent immunoassay, a protein A immunoassay, and an immunoelectrophoresis assay.

49. A method for detection of Helicobacter bizzozeronii in a sample of tissue or body fluids comprising:
providing an antibody or binding portion thereof according to claim 39;
contacting the sample with the antibody or binding portion thereof; and
detecting any reaction which indicates that Helicobacter bizzozeronii is present in the sample using an assay system.

50. A method according to claim 49, wherein said antibody is monoclonal or polyclonal.

51. A method for detection of Helicobacter bizzozeronii in a sample of tissue or body fluids comprising:
providing a nucleotide sequence of the nucleic acid molecule according to claim 3 as a probe in a nucleic acid hybridization assay;
contacting the sample with the probe; and
detecting any reaction which indicates that Helicobacter bizzozeronii is present in the sample.

52. A method for detection of Helicobacter bizzozeronii in a sample of tissue or body fluids comprising:
providing a nucleotide sequence of the nucleic acid molecule according to claim 3 as a probe in a gene amplification detection procedure;
contacting the sample with the probe; and
detecting any reaction which indicates that Helicobacter bizzozeronii is present in the sample.
